(19) United States
(12) Patent Application Publication
(10) Pub. No.: US 2026/0248692 A1
(43) Pub. Date: Aug. 27, 2026

(54) DENTAL ADHESIVE KIT AND METHOD FOR APPLYING THE SAME

(71) Applicant: SHOFU INC., Kyoto (JP)

(72) Inventors: Kenzo YAMAMOTO, Kyoto (JP); Rei NISHIMURA, Kyoto (JP); Toko FURUTANI, Kyoto (JP)

(73) Assignee: SHOFU INC., Kyoto (JP)

(21) Appl. No.: 19/421,236

(22) Filed: Dec. 16, 2025

(30) Foreign Application Priority Data
Feb. 27, 2025 (JP) 2025-030178

(51) Int. Cl. A61K 6/30; A61K 6/62; A61K 6/74; C09J 11/04; C09J 11/06; C09J 133/08; C09J 133/10; C09J 133/26

(52) U.S. Cl. CPC A61K 6/30; A61K 6/62; A61K 6/74; C09J 11/04; C09J 11/06; C09J 133/08; C09J 133/10; C09J 133/26

(57) ABSTRACT

A dental adhesive kit is provided. The dental adhesive kit includes: a dental adhesive composition (I) including a polymerizable monomer (A1) having an acidic group which is a polymerizable monomer (A), water (B), a volatile organic solvent (C), and a first photopolymerization initiator (D) comprising an aromatic tertiary amine compound (D1); and dental photocurable composition (II) including the polymerizable monomer (A), a second photopolymerization initiator (D′) comprising an α-diketone compound (D2), an iodonium salt compound (D3), and an aliphatic tertiary amine compound (D4) having an aromatic ring, and substantially free of the aromatic tertiary amine compound (D1), and a filler (E).

### TECHNICAL FIELD

The present disclosure relates to a dental adhesive kit and a method for applying the same.

### BACKGROUND ART

In dental treatment, dental adhesive kits including dental adhesive compositions and dental photocurable compositions are used. The dental photocurable compositions to be combined with dental adhesive compositions include dental composite resins, dental resin cements, dental core build-up materials, dental orthodontic materials, dental sealant materials, and dental splinting materials. Among these, dental orthodontic materials, dental sealant materials, and dental splinting materials are usually combined with pretreatment agents because high durable adhesive strength to enamel is required.

JP-A-2009-215254 discloses a dental filling/restoration kit, and WO-A-2022/030642 discloses a composition suitable for an aligner attachment.

### RELATED ART DOCUMENT

### Patent Document

Patent Document 1: JP-A-2009-215254
Patent Document 2: WO-A-2022/030642

### SUMMARY

### Problems to be Solved by the Disclosure

However, there is room for improvement in compatibility between high durable adhesive strength and color tone stability when these compositions are used for splinting materials or dental orthodontic adhesive kits.

An object of the present disclosure is to provide a dental adhesive kit that has both high durable adhesive strength and color tone stability.

### Means for Solving the Problems

As a result of elaborate studies, the inventors of the present disclosure have found that the above problem is solved by a predetermined dental adhesive kit.

The present disclosure provides the following items.
[1] A dental adhesive kit comprising:
   a dental adhesive composition (I) comprising a polymerizable monomer (A1) having an acidic group which is a polymerizable monomer (A), water (B), a volatile organic solvent (C), and a first photopolymerization initiator (D) comprising an aromatic tertiary amine compound (D1); and
   a dental photocurable composition (II) comprising the polymerizable monomer (A), a second photopolymerization initiator (D′) comprising an α-diketone compound (D2), an iodonium salt compound (D3), and an aliphatic tertiary amine compound (D4) having an aromatic ring, and substantially free of the aromatic tertiary amine compound (D1), and a filler (E).
[2] The dental adhesive kit according to [1], wherein the dental adhesive composition (I) further comprises a compound (F) comprising a disulfide group.
[3] The dental adhesive kit according to [2], wherein the compound (F) comprising a disulfide group is thioctic acid.
[4] The dental adhesive kit according to [1] or [2], wherein the dental adhesive composition (I) is substantially free of an aliphatic tertiary amine compound.
[5] The dental adhesive kit according to [3], wherein the dental adhesive composition (I) is substantially free of an aliphatic tertiary amine compound.
[6] The dental adhesive kit according to [1] or [2], wherein the dental adhesive composition (I) comprises,
   with respect to 100 parts by mass of the dental adhesive composition (I),
   3 to 20 parts by mass of the polymerizable monomer (A1) having an acidic group,
   10 to 50 parts by mass of the water (B),
   10 to 60 parts by mass of the volatile organic solvent (C), and
   0.05 to 2 parts by mass of the aromatic tertiary amine compound (D1), and
   the dental photocurable composition (II) comprises,
   with respect to 100 parts by mass of the polymerizable monomer (A) in the dental photocurable composition (II),
   0.05 to 0.6 parts by mass of the α-diketone compound (D2),
   0.3 to 5 parts by mass of the iodonium salt compound (D3),
   0.5 to 5 parts by mass of the aliphatic tertiary amine compound (D4) having an aromatic ring, and
   10 to 400 parts by mass of the filler (E).
[7] The dental adhesive kit according to [3], wherein the dental adhesive composition (I) comprises,
   with respect to 100 parts by mass of the dental adhesive composition (I),
   3 to 20 parts by mass of the polymerizable monomer (A1) having an acidic group,
   10 to 50 parts by mass of the water (B),
   10 to 60 parts by mass of the volatile organic solvent (C), and
   0.05 to 2 parts by mass of the aromatic tertiary amine compound (D1), and
   the dental photocurable composition (II) comprises,
   with respect to 100 parts by mass of the polymerizable monomer (A) in the dental photocurable composition (II),
   0.05 to 0.6 parts by mass of the α-diketone compound (D2),
   0.3 to 5 parts by mass of the iodonium salt compound (D3),
   0.5 to 5 parts by mass of the aliphatic tertiary amine compound (D4) having an aromatic ring, and
   10 to 400 parts by mass of the filler (E).
[8] The dental adhesive kit according to [4], wherein the dental adhesive composition (I) comprises,
   with respect to 100 parts by mass of the dental adhesive composition (I),
   3 to 20 parts by mass of the polymerizable monomer (A1) having an acidic group,
   10 to 50 parts by mass of the water (B),
   10 to 60 parts by mass of the volatile organic solvent (C), and
   0.05 to 2 parts by mass of the aromatic tertiary amine compound (D1), and
   the dental photocurable composition (II) comprises,
   with respect to 100 parts by mass of the polymerizable monomer (A) in the dental photocurable composition (II),
   0.05 to 0.6 parts by mass of the α-diketone compound (D2),
   0.3 to 5 parts by mass of the iodonium salt compound (D3),
   0.5 to 5 parts by mass of the aliphatic tertiary amine compound (D4) having an aromatic ring, and
   10 to 400 parts by mass of the filler (E).
[9] The dental adhesive kit according to [5], wherein the dental adhesive composition (I) comprises,
   with respect to 100 parts by mass of the dental adhesive composition (I),
   3 to 20 parts by mass of the polymerizable monomer (A1) having an acidic group,
   10 to 50 parts by mass of the water (B),
   10 to 60 parts by mass of the volatile organic solvent (C), and
   0.05 to 2 parts by mass of the aromatic tertiary amine compound (D1), and
   the dental photocurable composition (II) comprises,
   with respect to 100 parts by mass of the polymerizable monomer (A) in the dental photocurable composition (II),
   0.05 to 0.6 parts by mass of the α-diketone compound (D2),
   0.3 to 5 parts by mass of the iodonium salt compound (D3),
   0.5 to 5 parts by mass of the aliphatic tertiary amine compound (D4) having an aromatic ring, and
   10 to 400 parts by mass of the filler (E).

The dental adhesive kit according to any one of [1] to [9], further comprising a dental etching agent.

A method for applying the dental adhesive kit according to any one of [1] to [10], the method comprising applying the dental adhesive kit while performing at least one of (i) splinting mobile teeth and (ii) orthodontic treatment for bonding to enamel.

### Effects of the Disclosure

According to the embodiment of the present disclosure, a dental adhesive kit that has both durable adhesive strength and color tone stability is provided.

### EMBODIMENTS OF THE DISCLOSURE

Dental adhesive kits are used, for example, for dental orthodontic treatment or for dental splinting. Dental orthodontic adhesive kits are materials that may be used for bonding appliances used in orthodontic treatment or for preparing attachments in orthodontic treatment with aligners in order to improve misalignment of teeth and occlusion. Fixed appliances such as brackets and bands used in dental orthodontic treatment are subject to heavy loads and therefore require materials with high durable adhesive strength. Since fixed appliances are usually bonded to unground enamel, the enamel surface is roughened using a phosphoric acid etching agent or the like before the bonding operation. On the other hand, since the phosphoric acid etching agent has the risk of roughening enamel more than necessary and results in excessive durable adhesive strength in some cases, it is preferable to use a dental adhesive composition such as a self-etching primer to reduce the risk of excessive decalcification and to simplify operation. In recent years, orthodontic treatment using aligners has been performed in addition to the traditional fixed appliances that have been the mainstream. In aligner orthodontic treatment, protrusions called attachments are prepared on the tooth surface to help ensure a secure fit of the aligner and to control tooth movement. It is preferable that attachments as well as fixed appliances have high durable adhesive strength to enamel. In addition, adhesive materials for dental splinting for securing mobile teeth are also known to require high durable adhesive strength to enamel as in orthodontic treatment.

Aromatic amine compounds are used as photopolymerization initiators in dental adhesive compositions and dental photocurable compositions. Aromatic amine compounds may contribute to the development of good durable adhesive strength when used in dental adhesive compositions due to their high activity and may contribute to the development of good mechanical properties when used in dental photocurable compositions. However, since compositions containing aromatic amine compounds tend to discolor easily and have poor color tone stability, it is sometimes difficult to blend large amounts of aromatic amine compounds to the extent that exhibits sufficient properties in compositions where esthetic properties are required. In particular, when dental orthodontic adhesive kits are used to prepare attachments for aligner orthodontic treatment, it is preferable that the attachments do not discolor over time.

On the other hand, aliphatic amine compounds are also used as photopolymerization initiators in dental adhesive compositions and dental photocurable compositions. When aliphatic amine compounds are used, the polymerization activity is lower than that of aromatic amine compounds, but depending on the type of compound, the compositions may be unlikely to discolor. However, a polymerizable monomer having an acidic group in a dental adhesive composition sometimes reacts with the aliphatic amine compound, resulting in lower durable adhesive strength.

As a result of elaborate studies, the inventors of the present disclosure have found a dental adhesive kit containing a dental adhesive composition and a dental photocurable composition, in which an aromatic tertiary amine compound with high photopolymerization activity is blended in the dental adhesive composition, and a photoacid generator and an aliphatic tertiary amine compound containing an aromatic ring are blended in the dental photocurable composition. This finding has led to completion of the present disclosure.

Modes for carrying out the present disclosure are specifically described below, but the present disclosure is not limited to these.

As used herein, the expression “x and/or y” (x and y are each a given configuration) is intended to mean at least one of x and y and mean the following three meanings: only x; only y; and x and y.

As used herein, the expression “X to Y” (X and Y are each a given number) is intended to encompass “preferably more than X” or “preferably less than Y” unless otherwise specified, in addition to the meaning of “X or more and Y or less.”

As used herein, the expression “X or more” (X is a given number) or “Y or less” (Y is a given number) is intended to encompass “preferably more than X” or “preferably less than Y.”

For numerical ranges described herein in steps, the upper or lower limit of the numerical range in one step may be combined as desired with the upper or lower limit of the numerical range in another step. In the numerical ranges described herein, the upper or lower limits of the numerical ranges may be replaced by values listed in the examples.

As used herein, room temperature and various physical properties and characteristic values are all values at 23° C. unless otherwise noted.

A dental adhesive kit according to one embodiment of the present disclosure (hereinafter also referred to as “the present dental adhesive kit”) comprises:
a dental adhesive composition (I) comprising a polymerizable monomer (A1) having an acidic group which is a polymerizable monomer (A), water (B), a volatile organic solvent (C), and a first photopolymerization initiator (D) comprising an aromatic tertiary amine compound (D1); and
a dental photocurable composition (II) comprising the polymerizable monomer (A), a second photopolymerization initiator (D′) comprising an α-diketone compound (D2), an iodonium salt compound (D3), and an aliphatic tertiary amine compound (D4) having an aromatic ring, and substantially free of the aromatic tertiary amine compound (D1), and a filler (E).

### < (A) Polymerizable Monomer>

Any known polymerizable monomers may be used without limitation as polymerizable monomer (A) (also referred to as “component (A)”) contained in dental adhesive composition (I) and dental photocurable composition (II) included in the present dental adhesive kit. Polymerizable monomer (A) includes a polymerizable monomer (A1) having an acidic group and/or a polymerizable monomer (A2) having no acidic group. In the polymerizable monomer described in the present embodiment, a polymerizable group exhibiting radical polymerizability is preferred. Specifically, from the viewpoint of easy radical polymerization, the polymerizable group is preferably (meth)acrylic group and/or (meth)acrylamide group. As used herein “(meth)acrylic” means acrylic and/or methacrylic, “(meth)acryloyl” means acryloyl and/or methacryloyl, “(meth)acrylate” means acrylate and/or methacrylate, and “(meth)acrylamide” means acrylamide and/or methacrylamide. A polymerizable monomer having a substituent at the α-position of a (meth)acrylic group and/or a (meth)acrylamide group may also be preferably used. Herein, a silane coupling agent having a polymerizable group and particles surface-treated with the silane coupling agent having a polymerizable group, and components (B) to (F) containing polymerizable groups are not classified as polymerizable monomer (A).

### [(A1) Polymerizable Monomer Having Acidic Group]

Dental adhesive composition (I) included in the present dental adhesive kit contains a polymerizable monomer (A1) having an acidic group (also referred to as “component (A1)”). As polymerizable monomer (A1) having an acidic group, any polymerizable monomer may be used without limitation as long as it has one or more polymerizable groups and at least one acidic group such as phosphoric acid group, pyrophosphoric acid group, thiophosphoric acid group, phosphonic acid group, sulfonic acid group, and carboxylic acid group. The inclusion of a polymerizable monomer having an acidic group may impart adhesiveness to a tooth substance and a prosthetic device.

Specific examples of a polymerizable monomer having a phosphoric acid group include one or more selected from 2-(meth)acryloyloxyethyl dihydrogen phosphate, 3-(meth)acryloyloxypropyl dihydrogen phosphate, 4-(meth)acryloyloxybutyl dihydrogen phosphate, 5-(meth)acryloyloxypentyl dihydrogen phosphate, 6-(meth)acryloyloxyhexyl dihydrogen phosphate, 7-(meth)acryloyloxyheptyl dihydrogen phosphate, 8-(meth)acryloyloxyoctyl dihydrogen phosphate, 9-(meth)acryloyloxynonyl dihydrogen phosphate, 10-(meth)acryloyloxydecyl dihydrogen phosphate, 11-(meth)acryloyloxyundecyl dihydrogen phosphate, 12-(meth)acryloyloxydodecyl dihydrogen phosphate, 16-(meth)acryloyloxyhexadecyl dihydrogen phosphate, 20-(meth)acryloyloxyicosyl dihydrogen phosphate, bis[2-(meth)acryloyloxyethyl]hydrogen phosphate, bis[4-(meth)acryloyloxybutyl]hydrogen phosphate, bis[6-(meth)acryloyloxyhexyl]hydrogen phosphate, bis[8-(meth)acryloyloxyoctyl]hydrogen phosphate, bis[9-(meth)acryloyloxynonyl]hydrogen phosphate, bis[10-(meth)acryloyloxydecyl]hydrogen phosphate, 1,3-di(meth)acryloyloxypropyl dihydrogen phosphate, 2-(meth)acryloyl oxyethylphenyl hydrogen phosphate, 2-(meth)acryloyloxyethyl-2-bromoethyl hydrogen phosphate, and bis[2-(meth)acryloyloxy-(1-hydroxymethyl)ethyl]hydrogen phosphate; acid chloride, alkali metal salt, and ammonium salt thereof; and (meth)acrylamide compound in which the ester bond of any of these compounds is substituted with an amide bond, and the like.

Specific examples of a polymerizable monomer having a pyrophosphoric acid group include one or more selected from bis[2-(meth)acryloyloxyethyl]pyrophosphate, bis[4-(meth)acryloyloxybutyl]pyrophosphate, bis[6-(meth)acryloyloxyhexyl]pyrophosphate, bis[8-(meth)acryloyloxyoctyl]pyrophosphate, bis[10-(meth)acryloyloxydecyl]pyrophosphate; acid chloride, alkali metal salt, and ammonium salt thereof; and (meth)acrylamide compound in which the ester bond of any of these compounds is substituted with an amide bond, and the like.

Specific examples of a polymerizable monomer having a thiophosphoric acid group include one or more selected from 2-(meth)acryloyloxyethyl dihydrogen thiophosphate, 3-(meth)acryloyloxypropyl dihydrogen thiophosphate, 4-(meth)acryloyloxybutyl dihydrogen thiophosphate, 5-(meth)acryloyloxypentyl dihydrogen thiophosphate, 6-(meth)acryloyloxyhexyl dihydrogen thiophosphate, 7-(meth)acryloyloxyheptyl dihydrogen thiophosphate, 8-(meth)acryloyloxyoctyl dihydrogen thiophosphate, 9-(meth)acryloyloxynonyl dihydrogen thiophosphate, 10-(meth)acryloyloxydecyl dihydrogen thiophosphate, 11-(meth)acryloyloxyundecyl dihydrogen thiophosphate, 12-(meth)acryloyloxydodecyl dihydrogen thiophosphate, 16-(meth)acryloyloxyhexadecyl dihydrogen thiophosphate, 20-(meth)acryloyloxyicosyl dihydrogen thiophosphate; acid chloride, alkali metal salt, and ammonium salt thereof; and (meth)acrylamide compound in which the ester bond of any of these compounds is substituted with an amide bond, and the like.

Specific examples of a polymerizable monomer having a phosphonic acid group include one or more selected from 2-(meth)acryloyloxyethylphenyl phosphonate, 5-(meth)acryloyloxypentyl-3-phosphonopropionate, 6-(meth)acryloyloxyhexyl-3-phosphonopropionate, 10-(meth)acryloyloxydecyl-3-phosphonopropionate, 6-(meth)acryloyloxyhexyl-3-phosphonoacetate, 10-(meth)acryloyloxydecyl-3-phosphonoacetate; acid chloride, alkali metal salt, and ammonium salt thereof; and (meth)acrylamide compound in which the ester bond of any of these compounds is substituted with an amide bond, and the like.

Specific examples of a polymerizable monomer having a sulfonic acid group include one or more selected from 2-(meth)acrylamide-2-methylpropanesulfonic acid, 2-sulfoethyl (meth)acrylate, and the like.

Polymerizable monomers having a carboxylic acid group are classified into a (meth)acrylic compound having one carboxy group in the molecule and a (meth)acrylic compound having a plurality of carboxy groups in the molecule. Specific examples of the (meth)acrylic compound having one carboxy group in the molecule include one or more selected from (meth)acrylic acid, N-(meth)acryloylglycine, N-(meth)acryloylaspartic acid, O-(meth)acryloyltyrosine, N-(meth)acryloyltyrosine, N-(meth)acryloylphenylalanine, N-(meth)acryloyl-p-aminobenzoic acid, N-(meth)acryloyl-o-aminobenzoic acid, p-vinylbenzoic acid, 2-(meth)acryloyloxybenzoic acid, 3-(meth)acryloyloxybenzoic acid, 4-(meth)acryloyloxybenzoic acid, N-(meth)acryloyl-5-aminosalicylic acid, N-(meth)acryloyl-4-aminosalicylic acid, 2-(meth)acryloyloxyethyl hydrogen succinate, 2-(meth)acryloyloxyethyl hydrogen phthalate, 2-(meth)acryloyloxyethyl hydrogen malate; acid halide thereof; and (meth)acrylamide compound in which the ester bond of any of these compounds is substituted with an amide bond, and the like. Specific examples of the (meth)acrylic compound having a plurality of carboxy groups in the molecule include one or more selected from 6-(meth)acryloyloxyhexane-1,1-dicarboxylic acid, 9-(meth)acryloyloxynonane-1,1-dicarboxylic acid, 10-(meth)acryloyloxydecane-1,1-dicarboxylic acid, 11-(meth)acryloyloxyundecane-1,1-dicarboxylic acid, 12-(meth)acryloyloxydodecane-1,1-dicarboxylic acid, 13-(meth)acryloyloxytridecane-1,1-dicarboxylic acid, 4-(meth)acryloyloxyethyltrimellitic acid, 4-(meth)acryloyloxybutyltrimellitic acid, 4-(meth)acryloyloxyhexyltrimellitic acid, 4-(meth)acryloyloxydecyltrimellitic acid, 2-(meth)acryloyloxyethyl-3′-(meth)acryloyloxy-2′-(3,4-dicarboxybenzoyloxy) propyl succinate; acid anhydride and acid halide thereof; and (meth)acrylamide compound in which the ester bond of any of these compounds is substituted with an amide bond, and the like.

### [(A2) Polymerizable Monomer Having No Acidic Group]

As polymerizable monomer (A2) having no acidic group (also referred to as “component (A2)”), any polymerizable monomer that has one or more polymerizable groups and has no acidic group may be used without limitation. Polymerizable monomers (A2) having no acidic group include one or more selected from a polymerizable monomer having one radical polymerizable group, a polymerizable monomer having two radical polymerizable groups, a polymerizable monomer having three or more radical polymerizable groups, and the like.

Among polymerizable monomers (A2) having no acidic group, specific examples of the polymerizable monomer having one radical polymerizable group include one or more selected from 2-hydroxyethyl (meth)acrylate, 3-hydroxypropyl (meth)acrylate, 4-hydroxybutyl (meth)acrylate, 2-hydroxypropyl (meth)acrylate, 2-hydroxybutyl (meth)acrylate, 6-hydroxyhexyl(meth)acrylate, 10-hydroxydecyl(meth)acrylate, propylene glycol mono(meth)acrylate, glycerol mono(meth)acrylate, erythritol mono(meth)acrylate, N-methylol (meth)acrylamide, N-hydroxyethyl (meth)acrylamide, N,N-(dihydroxyethyl) (meth)acrylamide, methyl (meth)acrylate, ethyl (meth)acrylate, propyl (meth)acrylate, isopropyl (meth)acrylate, butyl (meth)acrylate, isobutyl (meth)acrylate, benzyl (meth)acrylate, lauryl (meth)acrylate, 2,3-dibromopropyl (meth)acrylate, 3-(meth)acryloyloxypropyltrimethoxysilane, 11-(meth)acryloyloxyundecyltrimethoxysilane, and (meth)acrylamide.

Among polymerizable monomers (A2) having no acidic group, specific examples of the polymerizable monomer having two radical polymerizable groups include one or more selected from 2,2-bis((meth)acryloyloxyphenyl) propane, 2,2-bis[4-(3-(meth)acryloyloxy)-2-hydroxypropoxyphenyl]propane (generally called “Bis-GMA”), 2,2-bis(4-(meth)acryloyloxyphenyl) propane, 2,2-bis(4-(meth)acryloyloxypolyethoxyphenyl) propane, 2,2-bis(4-(meth)acryloyloxydiethoxyphenyl) propane, 2,2-bis(4-(meth)acryloyloxytetraethoxyphenyl) propane, 2,2-bis(4-(meth)acryloyloxypentaethoxyphenyl) propane, 2,2-bis(4-(meth)acryloyloxydipropoxyphenyl) propane, 2-(4-(meth)acryloyloxy diethoxyphenyl)-2-(4-(meth)acryloyloxydiethoxyphenyl) propane, 2-(4-(meth)acryloyloxydiethoxyphenyl)-2-(4-(meth)acryloyloxyditriethoxyphenyl) propane, 2-(4-(meth)acryloyloxydipropoxyphenyl)-2-(4-(meth)acryloyloxytriethoxyphenyl) propane, 2,2-bis(4-(meth)acryloyloxypropoxyphenyl) propane, 2,2-bis(4-(meth)acryloyloxyisopropoxyphenyl) propane, 1,4-bis(2-(meth)acryloyloxyethyl) pyromellitate, glycerol di(meth)acrylate, 1-(acryloyloxy)-3-(methacryloyloxy)-2-propanol, ethylene glycol di(meth)acrylate, diethylene glycol di(meth)acrylate, triethylene glycol di(meth)acrylate, propylene glycol di(meth)acrylate, butylene glycol di(meth)acrylate, neopentyl glycol di(meth)acrylate, polyethylene glycol di(meth)acrylate, 1,3-butanediol di(meth)acrylate, 1,5-pentanediol di(meth)acrylate, 1,6-hexanediol di(meth)acrylate, 1,10-decanediol di(meth)acrylate, 1,2-bis(3-methacryloyloxy-2-hydroxypropoxy) ethane, 2,2,4-trimethylhexamethylene bis(2-carbamoyloxyethyl)dimethacrylate (generally called “UDMA”), and 1,2-bis(3-methacryloyloxy-2-hydroxypropoxy) ethane.

Among polymerizable monomers (A2) having no acidic group, specific examples of the polymerizable monomer having three radical polymerizable groups include one or more selected from trimethylolpropane tri(meth)acrylate, trimethylolethane tri(meth)acrylate, trimethylolmethane tri(meth)acrylate, pentaerythritol tri(meth)acrylate, pentaerythritol tetra(meth)acrylate, dipentaerythritol penta(meth)acrylate, N,N-(2,2,4-trimethylhexamethylene)bis[2-(aminocarboxy) propane-1,3-diol]tetramethacrylate, 1,7-diacryloyloxy-2,2,6,6-tetraacryloyloxymethyl-4-oxyheptane, and the like.

An oligomer or a prepolymer having at least one polymerizable group in the molecule may be used other than these polymerizable monomers, without any limitation. There is no problem if a substituent such as a fluoro group is present in the same molecule. The polymerizable monomers described above may be used singly or in combination of two or more.

Dental adhesive composition (I) included in the present dental adhesive kit contains polymerizable monomer (A1) having an acidic group. Preferable examples of polymerizable monomer (A1) having an acidic group that may be used include one or more selected from 10-methacryloyloxydecyl dihydrogen phosphate, 6-methacryloxyhexyl phosphonoacetate, 4-methacryloxyethyltrimellitic acid, and 4-methacryloxyethyltrimellitic anhydride. The preferred amount of polymerizable monomer (A1) having an acidic group that may be blended is 3 to 20 parts by mass with respect to 100 parts by mass of dental adhesive composition (I). When polymerizable monomer (A1) is present in an amount equal to or more than the above lower limit, good durable adhesive strength to enamel is exhibited. When polymerizable monomer (A1) is present in an amount equal to or less than the above upper limit, good storage stability is achieved. The preferred amount of polymerizable monomer (A) in dental adhesive composition (I) is 60 parts by mass or less and more preferably 50 parts by mass or less with respect to 100 parts by mass of dental adhesive composition (I). In such cases, the film thickness of dental adhesive composition (I) tends to become thinner, and esthetic treatment may be expected.

### < (B) Water>

Dental adhesive composition (I) included in the present dental adhesive kit contains water (B) (also referred to as “component (B)”). Specific examples of water (B) include one or more selected from deionized water and distilled water. The amount of water (B) is preferably 10 to 50 parts by mass with respect to 100 parts by mass of dental adhesive composition (I). When the amount of water (B) is equal to or more than the above lower limit, the durable adhesive strength to enamel tends to be good. When the amount of water (B) is equal to or less than the above upper limit, the solubility of polymerizable monomer (A) contained in dental adhesive composition (I) may be good.

### < (C) Volatile Organic Solvent>

Dental adhesive composition (I) included in the present dental adhesive kit may contain a volatile organic solvent (C) (also referred to as “component (C)”). Component (C) is usually an organic solvent with a boiling point of 150° C. or lower under normal pressure and a solubility in water of 5% by mass or more at 25° C., more preferably 30% by mass or more, and most preferably soluble in water at any rate. In particular, a water-soluble volatile organic solvent with a boiling point of 100° C. or lower under normal pressure is preferred, and specific examples include one or more selected from ethanol, methanol, 1-propanol, isopropyl alcohol, acetone, methyl ethyl ketone, 1,2-dimethoxyethane, 1,2-diethoxyethane, and tetrahydrofuran. Among the volatile organic solvents listed above, one or more selected from ethanol, isopropyl alcohol, acetone, and methyl ethyl ketone are even more preferred. The amount of component (C) is preferably 10 to 60 parts by mass with respect to 100 parts by mass of dental adhesive composition (I). When the amount of component (C) is equal to or more than the above lower limit, the film thickness tends to be thin when dental adhesive composition (I) is applied to an adherend surface, esthetic treatment may be expected, and the solubility of polymerizable monomer (A) contained in dental adhesive composition (I) may be good. When the amount of component (C) is equal to or less than the above upper limit, the balance of the formulation with other components is good in many cases.

### < (D) Photopolymerization Initiator>

Dental adhesive composition (I) and dental photocurable composition (II) included in the present dental adhesive kit contain a photopolymerization initiator (D) or (D′) (also referred to as “component (D)” or “component (D′)”). The photopolymerization initiator is a polymerization initiator capable of initiating polymerization by light irradiation. The photopolymerization initiator that may be used in the present dental adhesive kit includes one or more selected from photosensitizers, photoacid generators, and photopolymerization accelerators. These are not particularly limited, and any known compounds commonly used may be used without any limitation. Dental adhesive composition (I) and dental photocurable composition (II) included in the present dental adhesive kit may contain one of the photopolymerization initiators listed above, or two or more in combination.

### [(D1) Aromatic Tertiary Amine Compound]

Dental adhesive composition (I) included in the present dental adhesive kit contains an aromatic tertiary amine compound (D1) (also referred to as “component (D1)”), and dental photocurable composition (II) is substantially free of aromatic tertiary amine compound (D1).

The aromatic amine compound refers to a compound in which one or more H's of ammonia (NH₃) are substituted with an aromatic ring. An aromatic amine compound in which one H of NH₃ is substituted with an aromatic ring may be classified into an aromatic primary amine compound. An aromatic amine compound in which one H of NH₃ is substituted with an aromatic ring and one H of remaining two H's is substituted with an aromatic ring or an alkyl group may be classified into an aromatic secondary amine compound. An aromatic amine compound in which one H of NH₃ is substituted with an aromatic ring and remaining two H's are substituted with an aromatic ring or an alkyl group may be classified into an aromatic tertiary amine compound.

Specific examples of the aromatic tertiary amine compound include one or more selected from N,N-dimethylaniline, N,N-diethylaniline, N,N-di-n-butylaniline, N,N-dibenzylaniline, p-N,N-dimethyl-toluidine, m-N,N-dimethyl-toluidine, p-N,N-diethyl-toluidine, p-bromo-N,N-dimethylaniline, m-chloro-N,N-dimethylaniline, p-dimethylaminobenzaldehyde, p-dimethylaminoacetophenone, p-dimethylaminobenzoic acid, p-dimethylaminobenzoic acid ethyl ester, p-dimethylaminobenzoic acid isoamyl ester, p-dimethylaminobenzoic acid 2-butoxyethyl, p-dimethylaminobenzoic acid 2-ethylhexyl, p-dimethylaminobenzoic acid amino ester, N,N-dimethylanthranilic acid methyl ester, N,N-dihydroxyethyl aniline, N,N-diisopropanol aniline, p-N,N-dihydroxyethyl-toluidine, p-N,N-diisopropanol-toluidine, p-dimethylaminophenyl alcohol, p-dimethylaminostyrene, N,N-dimethyl-3,5-xylidine, 4-dimethylaminopyridine, N,N-dimethyl-α-naphthylamine, N,N-dimethyl-β-naphthylamine, and the like.

Dental adhesive composition (I) included in the present dental adhesive kit contains 0.05 to 2 parts by mass of component (D1) with respect to 100 parts by mass of dental adhesive composition (I). When component (D1) is present in an amount equal to or more than the above lower limit, good durable adhesive strength tends to be exhibited. When component (D1) is present in an amount equal to or less than the upper limit, storage stability tends to be good. The reason for the good durable adhesive strength is presumably as follows. Since component (D1) has high radical polymerization activity when combined with α-diketone compound (D2), light for curing dental photocurable composition (II) by light irradiation may cause the development of high curability at the interface of dental adhesive composition (I) in contact with dental photocurable composition (II), resulting in good durable adhesive strength. Specific examples of preferred component (D1) include one or more selected from p-dimethylaminobenzoic acid ethyl ester, p-dimethylaminobenzoic acid isoamyl ester, 1-cyano-4-(dimethylamino)benzene, p-N,N-dihydroxyethyl-toluidine, p-N,N-diisopropanol-toluidine, and the like. Among these, one or more selected from components (D1) having a dialkylamino group and an electron-withdrawing group such as ester group or cyano group at the 1- and 4-positions, such as p-dimethylaminobenzoic acid ethyl ester, p-dimethylaminobenzoic acid isoamyl ester, and 1-cyano-4-(dimethylamino)benzene, are preferred.

Dental photocurable composition (II) included in the present dental adhesive kit is substantially free of component (D1).

“Substantially free” means not intentionally blended, and when it is present as an impurity or a trace ingredient of a raw material, it does not correspond to “substantially free.” More specifically, the content of component (D1) is 0.1% by mass or less of dental photocurable composition (II), more preferably 0.05% by mass or less, even more preferably 0.01% by mass or less, and most preferably 0% by mass. The composition containing component (D1) tends to discolor when exposed to light for a long time. It is known to suppress the discoloration by blending a UV absorber or the like. However, UV absorbers are generally not effective enough to improve the mechanical properties of the composition, so it is preferable that a UV absorber is not blended in a large amount, such as 2 or more parts by mass in 100 parts by mass of the composition. Dental adhesive composition (I) of the present dental adhesive kit has a thin film thickness after being applied to an adherend and air-dried. The thickness of dental adhesive composition (I) is usually 50 μm or less, preferably 20 μm or less, and even more preferably 5 μm or less. Therefore, even if discoloration occurs due to the inclusion of component (D1), the discoloration is less noticeable. On the other hand, dental photocurable composition (II) is usually used in a thickness of 1 mm or more. Therefore, if the color tone of its cured product changes, the color change is easily noticeable. It is believed that the present dental adhesive kit acquires good durable adhesive strength by blending component (D1) in dental adhesive composition (I), and is less likely to discolor and has excellent esthetic properties because dental photocurable composition (II) is substantially free of component (D1).

### [(D2) α-Diketone Compound]

Dental photocurable composition (II) included in the present dental adhesive kit contains an α-diketone compound (D2) (also referred to as “component (D2)”). α-Diketone compound (D2) refers to a diketone compound with a structure in which two ketone groups are adjacent to each other. Such compounds include benzyl, camphorquinone, camphorquinone carboxylic acid, and camphorquinone sulfonic acid. Component (D2) may be selected as appropriate according to the wavelength, the intensity, and the irradiation time of light used for polymerization, and the type and the amount of other components to be combined. Photosensitizers may be used singly or in combination of two or more. Among components (D2), α-diketone compounds having a maximum absorption wavelength in the visible light region are preferably used, and one or more camphorquinone compounds selected from camphorquinone, camphorquinone carboxylic acid, camphorquinone sulfonic acid, and the like are more preferred. Camphorquinone is particularly preferred because it is easily available.

The preferred amount of component (D2) of dental photocurable composition (II) included in the present dental adhesive kit is preferably 0.05 to 0.6 parts by mass with respect to 100 parts by mass of the total amount of polymerizable monomer (A) contained in dental photocurable composition (II). When the amount of photosensitizer is equal to or more than the above lower limit, polymerization activity for irradiation light tends to be fully exhibited, and good curability tends to be achieved. When the amount of photosensitizer is equal to or less than the above upper limit, ambient light stability is good, and furthermore, esthetic properties tend to be good due to low yellow tone.

### [(D3) Iodonium Salt Compound]

Dental photocurable composition (II) included in the present dental adhesive kit contains an iodonium salt compound (D3) (also referred to as “component (D3)”). Any known component (D3) may be used. As a specific example, the structural formula of component (D3) may be represented by formula (1).

[(R1)₂I]⁺[A]⁻

(In the formula, [(R1)₂I]⁺ is a cation part, [A]⁻ is an anion part, R1 in formula (1) represents an organic group bonded to I, and R1s may be the same or different. R1 represents, for example, an aryl group having 6 to 30 carbon atoms, a heterocyclic group having 4 to 30 carbon atoms, an alkyl group having 1 to 30 carbon atoms, an alkenyl group having 2 to 30 carbon atoms, or an alkynyl group having 2 to 30 carbon atoms, and these may have at least one substituent selected from the group consisting of alkyl, hydroxy, alkoxy, alkylcarbonyl, arylcarbonyl, alkoxycarbonyl, aryloxycarbonyl, arylthiocarbonyl, acyloxy, arylthio, alkylthio, aryl, heterocyclic, aryloxy, alkylsulfinyl, arylsulfinyl, alkylsulfonyl, arylsulfonyl, alkyleneoxy, amino, cyano, and nitro groups, and halogens.)

It should be noted that I in the formula is different from (I) in the present document, and I in the formula is the elemental symbol for iodine.

In the above, examples of the aryl group having 6 to 30 carbon atoms include one or more selected from monocyclic aryl groups such as phenyl group, one or more condensed polycyclic aryl groups selected from naphthyl, anthracenyl, phenanthrenyl, pyrenyl, chrysenyl, naphthacenyl, benzanthracenyl, anthraquinolyl, fluorenyl, naphthoquinone, and anthraquinone, and the like.

Examples of the heterocyclic group having 4 to 30 carbon atoms include cyclic groups containing 1 to 3 heteroatoms such as oxygen, nitrogen, and sulfur, which may be the same or different. Specific examples include one or more selected from one or more monocyclic heterocyclic groups selected from thienyl, furanyl, pyranyl, pyrrolyl, oxazolyl, thiazolyl, pyridyl, pyrimidyl, pyrazinyl, and the like, and one or more condensed polycyclic heterocyclic groups selected from indolyl, benzofuranyl, isobenzofuranyl, benzothienyl, isobenzothienyl, quinolyl, isoquinolyl, quinoxalinyl, quinazolinyl, carbazolyl, acridinyl, phenothiazinyl, phenazinyl, xanthenyl, thianthrenyl, phenoxazinyl, phenoxathiinyl, chromanyl, isochromanyl, dibenzothienyl, xanthenyl, thioxanthenyl, dibenzofuranyl, and the like.

Specific examples of the alkyl group having 1 to 30 carbon atoms include one or more selected from one or more linear alkyl groups selected from methyl, ethyl, propyl, butyl, hexadecyl, octadecyl, and the like, one or more branched alkyl groups selected from isopropyl, isobutyl, sec-butyl, tert-butyl, isopentyl, neopentyl, tert-pentyl, isohexyl, and the like, and one or more cycloalkyl groups selected from cyclopropyl, cyclobutyl, cyclopentyl, cyclohexyl, and the like.

Specific examples of the alkenyl group having 2 to 30 carbon atoms include one or more linear or branched groups selected from vinyl, allyl, 1-propenyl, isopropenyl, 1-butenyl, 2-butenyl, 3-butenyl, 1-methyl-1-propenyl, and the like.

Specific examples of the alkynyl group having 2 to 30 carbon atoms include one or more linear or branched groups selected from ethynyl, 1-propynyl, 2-propynyl, 1-butynyl, 2-butynyl, 3-butynyl, 1-methyl-1-propynyl, 1-methyl-2-propynyl, and the like.

The aryl group having 6 to 30 carbon atoms, the heterocyclic group having 4 to 30 carbon atoms, the alkyl group having 1 to 30 carbon atoms, the alkenyl group having 2 to 30 carbon atoms, or the alkynyl group having 2 to 30 carbon atoms may have at least one substituent. Specific examples of the substituent include one or more selected from: one or more linear alkyl groups having 1 to 18 carbon atoms selected from methyl, ethyl, propyl, butyl, octadecyl, and the like; one or more branched alkyl groups having 1 to 18 carbon atoms selected from isopropyl, isobutyl, sec-butyl, tert-butyl, and the like; one or more cycloalkyl groups having 3 to 18 carbon atoms selected from cyclopropyl, cyclobutyl, cyclopentyl, cyclohexyl, and the like; hydroxy groups; one or more linear or branched alkoxy groups having 1 to 18 carbon atoms selected from methoxy, ethoxy, propoxy, isopropoxy, butoxy, isobutoxy, sec-butoxy, tert-butoxy, dodecyloxy, and the like; one or more linear or branched alkylcarbonyl groups having 2 to 18 carbon atoms selected from acetyl, propionyl, butanoyl, 2-methylpropionyl, heptanoyl, 2-methylbutanoyl, 3-methylbutanoyl, octanoyl, and the like; one or more arylcarbonyl groups having 7 to 11 carbon atoms selected from benzoyl, naphthoyl, and the like; one or more linear or branched alkoxycarbonyl groups having 2 to 19 carbon atoms selected from methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, and the like; one or more aryloxycarbonyl groups having 7 to 11 carbon atoms selected from phenoxycarbonyl, naphthoxycarbonyl, and the like; one or more arylthiocarbonyl groups having 7 to 11 carbon atoms selected from phenylthiocarbonyl, naphthoxythiocarbonyl, and the like; one or more linear or branched acyloxy groups having 2 to 19 carbon atoms selected from acetoxy, ethylcarbonyloxy, propylcarbonyloxy, isobutylcarbonyloxy, sec-butylcarbonyloxy, tert-butylcarbonyloxy, octadecylcarbonyloxy, and the like; one or more arylthio groups having 6 to 20 carbon atoms selected from phenylthio, biphenylylthio, methylphenylthio, chlorophenylthio, bromophenylthio, fluorophenylthio, hydroxyphenylthio, methoxyphenylthio, naphthylthio, 4-[4-(phenylthio)benzoyl]phenylthio, 4-[4-(phenylthio) phenoxy]phenylthio, 4-[4-(phenylthio)phenyl]phenylthio, 4-(phenylthio)phenylthio, 4-benzoylphenylthio, 4-benzoyl-chlorophenylthio, 4-benzoyl-methylthiophenylthio, 4-(methylthiobenzoyl)phenylthio, 4-(tert-butylbenzoyl)phenylthio, and the like; one or more linear or branched alkylthio groups having 1 to 18 carbon atoms selected from methylthio, ethylthio, propylthio, tert-butylthio, neopentylthio, dodecylthio, and the like; one or more aryl groups having 6 to 10 carbon atoms selected from phenyl, tolyl, dimethylphenyl, naphthyl, and the like; one or more heterocyclic groups having 4 to 20 carbon atoms selected from thienyl, furanyl, pyranyl, xanthenyl, chromanyl, isochromanyl, xanthonyl, thioxanthenyl, dibenzofuranyl, and the like; one or more aryloxy groups having 6 to 10 carbon atoms selected from phenoxy, naphthyloxy, and the like; one or more linear or branched alkylsulfinyl groups having 1 to 18 carbon atoms selected from methylsulfinyl, ethylsulfinyl, propylsulfinyl, tert-pentylsulfinyl, octylsulfinyl, and the like; one or more arylsulfinyl groups having 6 to 10 carbon atoms selected from phenylsulfinyl, tolylsulfinyl, naphthylsulfinyl, and the like; one or more linear or branched alkylsulfonyl groups having 1 to 18 carbon atoms selected from methylsulfonyl, ethylsulfonyl, propylsulfonyl, isopropylsulfonyl, butylsulfonyl, octylsulfonyl, and the like; one or more arylsulfonyl groups having 6 to 10 carbon atoms selected from phenylsulfonyl, tolylsulfonyl (tosyl), naphthylsulfonyl, and the like; alkyleneoxy groups; cyano groups; nitro groups; and one or more halogens selected from fluorine, chlorine, bromine, iodine, and the like.

Among the iodonium salt compounds, aryl iodonium salt is preferred because of its high stability. Further, it is preferable that the aryl group has a substituent in order to improve fat solubility. Specifically, one or more selected from one or more linear alkyl groups selected from methyl, propyl, octyl, decyl, undecyl, dodecyl, tridecyl, and the like, one or more branched alkyl groups selected from isopropyl, isobutyl, sec-butyl, tert-butyl, isopentyl, neopentyl, tert-pentyl, isohexyl, and the like, or functional groups in which one or more H's of these hydrocarbon groups are substituted with F, perfluoroalkyl groups, halogens, and the like are preferred as the substituent.

The structure of the anion part of the iodonium salt compound is not particularly limited, and examples include those having atoms such as halogens, P, S, B, Al, and Ga. It is preferable that the anions of these atoms have organic groups such as alkyl and/or alkoxy and/or aryl groups. In this case, when the anion part has an organic group such as alkyl and/or alkoxy and/or aryl groups in which one or more H's of the organic group are substituted with F, the solubility in the dental photocurable composition becomes high, which helps to prevent precipitation during low-temperature storage or long-term storage and to shorten the time taken for production due to dissolution in the composition in a short time. It is not preferable if the iodonium salt compound has low solubility and is precipitated, because the color tone stability and flexural strength of the dental photocurable composition may be reduced.

Since the anion part [A]⁻ of the iodonium salt compound represented by formula (1) improves the solubility in the dental photocurable composition, it is preferable that the anion has an organic group such as alkyl and/or alkoxy and/or aryl groups in which at least one H is substituted with F. Specifically, the number of carbon atoms of the alkyl group in the anion part [A]⁻ of the iodonium salt compound represented by formula (1) is preferably 1 to 8, and more preferably 1 to 4. Specific examples include one or more selected from one or more linear alkyl groups selected from methyl, ethyl, propyl, butyl, pentyl, octyl, and the like; one or more branched alkyl groups selected from isopropyl, isobutyl, sec-butyl, tert-butyl, and the like; and one or more cycloalkyl groups selected from cyclopropyl, cyclobutyl, cyclopentyl, cyclohexyl, and the like. The ratio (F/H) of the number of fluorine atoms to the number of hydrogen atoms in the alkyl group is 4 or more, and the ratio (F/H) of the number of fluorine atoms to the number of hydrogen atoms in the alkyl group is preferably 9 or more. More preferably, all hydrogen atoms of the hydrocarbon are substituted with fluorine. An iodonium salt including an anion having an alkyl group with a different ratio of hydrogen atoms and fluorine atoms may be blended in the dental photocurable composition.

Furthermore, specific examples of the alkyl group include one or more linear or branched perfluoroalkyl groups selected from CF₃, CF₃CF₂, (CF₃)₂CF, CF₃CF₂CF₂, CF₃CF₂CF₂CF₂, (CF₃)₂CFCF₂, CF₃CF₂ (CF₃) CF, (CF₃)₃C, and the like.

The number of carbon atoms of the alkoxy group in the anion part [A]⁻ of the iodonium salt compound represented by formula (1) is 1 to 8, and preferably 1 to 4. Specific examples include one or more linear alkoxy groups selected from methoxy, ethoxy, propoxy, butoxy, pentoxy, octoxy, and the like, and one or more branched alkoxy groups selected from isopropoxy, isobutoxy, sec-butoxy, tert-butoxy, and the like. The ratio (F/H) of the number of fluorine atoms to the number of hydrogen atoms in the alkyl group is 4 or more, and the ratio (F/H) of the number of fluorine atoms to the number of hydrogen atoms in the alkyl group is preferably 9 or more. More preferably, all hydrogen atoms of the hydrocarbon are substituted with fluorine. An iodonium salt including an anion having an alkoxy group with a different ratio of hydrogen atoms and fluorine atoms may be blended in the dental photocurable composition.

Furthermore, specific examples of the alkoxy group include one or more linear or branched perfluoroalkoxy groups selected from CF₃O, CF₃CF₂O, CF₃CF₂CF₂O, (CF₃)₂CFO, CF₃CF₂CF₂CF₂O, (CF₃)₂CFCF₂O, CF₃CF₂ (CF₃) CFO, CF₃CF₂CF₂CF₂CF₂O, CF₃CF₂CF₂CF₂CF₂CF₂CF₂CF₂CF₂O, and the like.

The phenyl group in the anion part [A]⁻ of the iodonium salt compound represented by formula (1) is a phenyl group in which at least one or more hydrogen atoms are substituted with fluorine, and/or an alkyl group and/or alkoxy group substituted with fluorine. The alkyl group and/or alkoxy group substituted with fluorine are preferably those listed above. Specific examples of particularly preferred phenyl groups include one or more perfluorophenyl groups selected from pentafluorophenyl (C₆F₅), trifluorophenyl (C₆H₂F₃), tetrafluorophenyl (C₆HF₄), trifluoromethylphenyl (CF₃C₆H₄), bis(trifluoromethyl)phenyl ((CF₃)₂C₆H₃), pentafluoroethylphenyl (CF₃CF₂C₆H₄), bis(pentafluoroethyl)phenyl ((CF₃CF₂)₂C₆H₃), trifluoromethyl fluorophenyl (CF₃C₆H₃F), bistrifluoromethyl fluorophenyl ((CF₃)₂C₆H₂F), pentafluoroethyl fluorophenyl (CF₃CF₂C₆H₃F), bispentafluoroethyl fluorophenyl ((CF₃CF₂)₂C₆H₂F), and the like. An iodonium salt including an anion having a phenyl group with a different ratio of hydrogen atoms and fluorine atoms may be blended in the dental photocurable composition.

As specific examples of the anion part [A]⁻ in the iodonium salt compound represented by formula (1), examples of anions having P include one or more selected from [(CF₃CF₂)₃PF₃]⁻, [(CF₃CF₂CF₂)₃PF₃]⁻, [((CF₃)₂CF)₂PF₄]⁻, [((CF₃)₂CF)₃PF₃]⁻, [((CF₃)₂CF)₄PF₂]⁻, [((CF₃)₂CFCF₂)₂PF₄]⁻, [((CF₃)₂CFCF₂)₃PF₃]⁻, and the like. Examples of anions having S include one or more selected from [(CF₃SO₂)₃C]⁻, [(CF₃CF₂SO₂)₃C]⁻, [(CF₃CF₂CF₂SO₂)₃C]⁻, [(CF₃CF₂CF₂CF₂SO₂)₃C], [CF₃CF₂CF₂CF₂SO₃], [CF₃CF₂CF₂SO₃]⁻, [(CF₃CF₂SO₂)₃C], [(SO₂CF₃)₃N]⁻, [(SO₂CF₂CF₃)₂N]⁻, [((CF₃) C₆H₄)SO₃]⁻, [SO₃((CF₂CF₂CF₂CF₂)SO₃]²⁻, and the like. Examples of anions having B include one or more selected from [B(C₆F₅)₄]⁻, [(C₆H₅)B((CF₃)₂C₆H₃)₃]⁻, [(C₆H₅)B(C₆F₅)₃]⁻, and the like. Examples of anions having Ga include one or more selected from [((C₆F₅)₃(C₆H₅)Ga)]⁻, [((C₆F₅)₃(C₄F₉)Ga)]⁻, [((C₆H₂F₃)₄Ga)]⁻, [((CF₃)₂C₆H₃)₄Ga)]⁻, [((CF₃)₄Ga)]⁻, [Ga(C₆F₅)₄]⁻, and the like. Examples of anions having Al include one or more selected from [((CF₃)₃CO)₄Al]⁻, [((CF₃CF₂)₃CO)₄Al]⁻, and the like.

Examples of other anions include one or more selected from one or more halogens selected from chloride, bromide, and the like, perhalogenoic acids such as perchloric acid, aromatic sulfonic acids such as p-toluenesulfonate, camphorsulfonic acids, nitrates, acetates, chloroacetates, carboxylates, phenolates, tetrafluoroborates, hexafluorophosphates, hexafluoroantimonates, hexafluoroarsenates, and the like. Among these, one or more selected from p-toluenesulfonates, camphorsulfonic acids, and carboxylates are preferably used.

The preferred amount of component (D3) in dental photocurable composition (II) included in the present dental adhesive kit is 0.3 to 5 parts by mass with respect to 100 parts by mass of polymerizable monomer (A) contained in dental photocurable composition (II). When component (D3) is present in an amount equal to or more than the above lower limit, good polymerization accelerating ability is exhibited. When component (D3) is present in an amount equal to or less than the above upper limit, good ambient light stability is exhibited, and yellow tone or brown tone in the initial stage of curing is low, resulting in good esthetic properties.

### [(D4) Aliphatic Tertiary Amine Compound Having Aromatic Ring]

Dental photocurable composition (II) included in the present dental adhesive kit contains an aliphatic tertiary amine compound (D4) having an aromatic ring (also referred to as “component (D4)”). Aliphatic tertiary amine compound (D4) having an aromatic ring is distinguished from aromatic tertiary amine compound (D1). When an aromatic ring is bonded to N in an amine compound, the amine compound corresponds to component (D1). The aliphatic tertiary amine compound refers to a compound in which N in the amine compound is bonded to carbon that does not form an aromatic ring, such as a methine or methylene group. Furthermore, aliphatic tertiary amine compound (D4) having an aromatic ring refers to a compound having an aromatic ring in the compound structure among aliphatic tertiary amine compounds, and refers to a compound with no aromatic ring bonded to N. In component (D4), the aromatic ring is preferably a benzyl group bonded to carbon at the α-position relative to the amine-derived N atom.

Dental photocurable composition (II) included in the present dental adhesive kit may exhibit good curability by containing photopolymerization initiator (D′) including component (D2), component (D3), and component (D4). Usually, N-methylethanolamine, dimethylaminoethyl methacrylate, and the like are used as aliphatic tertiary amine compounds in the dental field. However, when such aliphatic tertiary amine compounds are used in a dental adhesive kit, durable adhesive strength may be reduced. This is particularly noticeable when dental adhesive composition (I) included in the dental adhesive kit is applied to an adherend surface, and then dental photocurable composition (II) is applied on dental adhesive composition (I) not photocured. In the present embodiment, it has been found that the use of component (D4) as an aliphatic tertiary amine compound provides good durable adhesive strength. This effect is considered to be due to the steric hindrance caused by the aromatic ring in component (D4), which prevents a neutralization reaction with polymerizable monomer (A1) having an acidic group in dental adhesive composition (I), resulting in good durable adhesive strength.

Specific examples of component (D4) include one or more selected from N,N-dimethylbenzylamine, N-ethyl-N-methylbenzylamine, N,N-dimethyl-1-phenylethylamine, N,N-dimethylaminomethylphenol, N-methyl-N-(2-propyn-1-yl)benzylamine, 3-benzyloxazolidine, N,N-diethylbenzylamine, 3-[1-(dimethylamino)ethyl]phenol, 1-benzyl-3-pyrrolidone, 1-benzylpiperidine, N-benzyl diethanolamine, N-cyanodibenzylamine, N-benzyliminodiacetic acid, N-benzyl-3,3′-iminodipropionic acid, 1-benzhydryl-3-azetidinone, 1-(diphenylmethyl)-3-azetidinol, N,N-dibenzylaminoethanol, N,N-dibenzylaminopropanol, 2,6-di-tert-butyl-4-dimethylaminomethylphenol, 2,4,6-tris(dimethylaminomethyl) phenol, dibenzylaminopropionaldehyde, tribenzylamine, dibenzylaminoethyl (meth)acrylate, dibenzylaminopropyl(meth)acrylate, benzyliminodiethyl (meth)acrylate, tris(2-picolyl)amine, N-(4-tert-butylbenzyl)-N-methyl-1-naphthalenemethanamine, 3-dibenzylamino-2-fluoropronaphthalenemethylamine benzylpionate, 9-[2-(dibenzylamino)ethyl]-6-methyl-2,3,4,9-tetrahydro-1H-carbazol-1-one, (S)-(−)-2-(dibenzylamino) propionaldehyde, (S)-(+)-2-(dibenzylamino)-1-propanol, (S)-2-(dibenzylamino)-3-methylbutanol, 1-[(dibenzylamino)methyl]-2-naphthanol, (2S)-2-(dibenzylamino)-4-methyl-1-pentanol, (S)-(+)-2-(dibenzylamino)-3-phenyl-1-propanol, and the like. Examples of those having a heterocyclic structure include one or more selected from N-benzoylmeroquinene tert-butyl ester, ethyl 1-benzyl-4-piperidinecarboxylate, N-benzylnortropinone, 1-benzyl-3-methyl-4-piperidone, 1-benzyl-4-piperidine carboxyaldehyde, 1-benzyl-4-piperidone, 1-benzylpiperidine, and the like. Other examples include one or more selected from benzyl group-protected amino acids and benzyl-protected amino acid esters, such as N,N-dibenzylglycine ethyl ester, N-benzoyl-L-tyrosine ethyl ester, benzoyl-DL-phenylalanine, benzoyl-DL-alanine, benzoyl-DL-methionine, benzoyl-DL-leucine, benzoyl-DL-valine, N-benzoyl-L-glutamic acid, N-benzoyl-DL-phenylalanine 2-naphthyl, N-benzoyl-L-tyrosine, and the like.

Among the compounds listed above, those having a benzyl group are preferred, and those having a dibenzyl group are more preferred. Specific examples include one or more selected from dibenzylaminoethyl (meth)acrylate, dibenzylaminopropyl (meth)acrylate, dibenzylaminoethanol, dibenzylaminopropanol, dibenzylmethylamine, N,N-dibenzylglycine ethyl ester, tribenzylamine, and the like.

Dental photocurable composition (II) included in the present dental adhesive kit preferably contains component (D4) in an amount of 0.5 to 5 parts by mass with respect to 100 parts by mass of the total amount of polymerizable monomer (A). When component (D4) is present in an amount equal to or more than the above lower limit, good curability tends to be exhibited. When component (D4) is present in an amount equal to or less than the above upper limit, ambient light stability tends to be good.

It is preferable that dental adhesive composition (I) included in the present dental adhesive kit is substantially free of an aliphatic tertiary amine compound. The inclusion of an aliphatic tertiary amine compound may reduce durable adhesive strength to enamel. “Substantially free” means not intentionally blended, and when it is present as an impurity or a trace ingredient of a raw material, it does not correspond to “substantially free.” More specifically, the amount of aliphatic tertiary amine compound is preferably 0.1 parts by mass or less, more preferably 0.01 parts by mass or less, and most preferably 0 parts by mass, with respect to 100 parts by mass of dental adhesive composition (I). Specific examples of the aliphatic tertiary amine compound which is preferably substantially absent include N-methylethanolamine and dimethylaminoethyl methacrylate.

Compounds contained in photopolymerization initiator (D) or (D′) may be a combination of a plurality of compounds.

### < (E) Filler>

Dental photocurable composition (II) included in the present dental adhesive kit contains a filler (E) (also referred to as “component (E)”). Examples of filler (E) include inorganic fillers, organic fillers, organic-inorganic composite fillers, and ion sustained-release glass. In dental photocurable composition (II) used in the present embodiment, the fillers described as examples may be used singly or in combination of two or more.

The chemical compositions of the inorganic fillers are not particularly limited, but specific examples include one or more selected from silicon dioxide, alumina, titania, silica-titania, silica-titania-barium oxide, silica-zirconia, silica-alumina, lanthanum glass, borosilicate glass, soda glass, barium glass, strontium glass, glass ceramic, aluminosilicate glass, barium boroaluminosilicate glass, strontium boroaluminosilicate glass, fluoroaluminosilicate glass, calcium fluoroaluminosilicate glass, strontium fluoroaluminosilicate glass, barium fluoroaluminosilicate glass, strontium calcium fluoroaluminosilicate glass, and the like. In particular, one or more selected from barium fluoroaluminosilicate glass, strontium fluoroaluminosilicate glass, fluoroaluminosilicate glass, and the like, which are used in dental glass ionomer cements, resin reinforced glass ionomer cements, resin cements, and the like, may be preferably used. As used herein, fluoroaluminosilicate glass has a backbone of silicon oxide and aluminum oxide and contains an alkali metal for introducing non-bridging oxygen. The fluoroaluminosilicate glass further has an alkaline earth metal including strontium and fluorine as modification/coordination ions. The fluoroaluminosilicate glass is a composition in which a lanthanoid series element is incorporated into the backbone in order to impart further radiopacity. This lanthanoid series element is also incorporated into the composition as a modification/coordination ion, depending on the composition range.

The inorganic fillers may include hydrophobized inorganic fine particles. The primary particles of the hydrophobized inorganic fine particles preferably have an average particle size of 0.1 to 50 nm, and the hydrophobization is preferably treatment with a silane coupling agent and/or a modified silicone oil. Blending the hydrophobized inorganic fine particles is expected to suppress the sedimentation of the inorganic filler and impart rheological properties, in addition to improving the flexural strength.

Specific examples of the organic fillers include one or more polymers selected from polymethyl methacrylate, polyethyl methacrylate, methyl methacrylate-ethyl methacrylate copolymer, ethyl methacrylate-butyl methacrylate copolymer, methyl methacrylate-trimethylolpropane methacrylate copolymer, polyvinylchloride, polystyrene, chlorinated polyethylene, nylon, polysulfone, polyethersulfone, polycarbonate, and the like.

Specific examples of the organic-inorganic composite fillers include, without limitation, one or more selected from those obtained by coating the surface of an inorganic filler with a polymerizable monomer by polymerization; those obtained by mixing an inorganic filler and a polymerizable monomer, polymerizing the monomer, and thereafter grinding the resultant to a proper particle size; those obtained by dispersing an inorganic filler in a polymerizable monomer in advance for emulsion polymerization or suspension polymerization; those obtained by dispersing an inorganic filler in a polymerizable monomer and a solvent in advance, and spray-drying and then polymerizing the dispersion; those obtained by dispersing an inorganic filler in a solvent in advance, spray-drying the dispersion, and then impregnating the resultant with a polymerizable monomer for polymerization, and the like.

The ion sustained-release glass is characterized in that at least one of fluorine ion, strontium ion, borate ion, and aluminum ion is sustainably released. Among these ions, it is preferable that a plurality of these ions are released simultaneously.

As the ion sustained-release glass used in the present embodiment, any ion sustained-release glass may be used without any limitation as long as such ion sustained-release glass includes one or more kinds of glass-network forming elements for forming a glass network, and one or more kinds of glass modifying elements for modifying the glass network. These ion sustained-release glasses may be used singly or in combination of two or more. In the present embodiment, a glass amphoteric element that serves both as a glass-network forming element and as a glass modifying element depending on the glass composition falls into the category of glass-network forming element. Specific examples of the glass-network forming element contained in the ion sustained-release glass include one or more selected from silica, aluminum, boron, phosphorus, and the like. Specific examples of the glass modifying element include one or more selected from one or more halogen elements selected from fluorine, bromine, iodine, and the like; one or more alkali metal elements selected from sodium, lithium, and the like; and one or more alkali earth metal elements selected from calcium, strontium, and the like. Among these, the ion sustained-release glass preferably includes one or more selected from silica, aluminum, boron, and the like, as the glass-network forming element, and includes one or more selected from fluorine, sodium, strontium, and the like, as the glass modifying element. Specific examples include one or more selected from silica glass, fluoroaluminosilicate glass, fluoroborosilicate glass, fluoroaluminoborosilicate glass, and the like that contain one or more selected from strontium, sodium, and the like. Furthermore, from the viewpoint of sustained release of one or more selected from fluorine ion, strontium ion, borate ion, aluminum ion, and the like, fluoroaluminoborosilicate glass containing strontium is more preferable. As a specific example, more preferred glass composition ranges are: 10 to 40% by mass of SiO₂, 10 to 35% by mass of Al₂O₃, 2.5 to 30% by mass of B₂O₃, 15 to 50% by mass of SrO, 2.5 to 20% by mass of F, and 0 to 15% by mass of Na₂O. The glass composition may be identified by using instrumental analysis such as elementary analysis, Raman spectrum, and fluorescence X-ray analysis, and there is no problem as long as the actual measurements by any analysis methods match these composition ranges.

These ion sustained-release glasses may be produced by any method without limitation and may be produced by a production method such as melting method or sol-gel method. In particular, a production method by a melting method using a melting furnace is preferred in terms of ease of design of the glass composition, including raw material selection. The ion sustained-release glass used in the present embodiment has an amorphous structure, but there is no problem even if it contains a partially crystalline structure. Further, a mixture of a glass having an amorphous structure and a glass having a crystal structure may be used without any problem. Whether the glass structure is amorphous may be determined by using X-ray diffraction analysis or using an analysis instrument such as transmission electron microscope. In particular, the ion sustained-release glass used in the present embodiment preferably has an amorphous structure, which is a homogeneous structure, because it sustainably releases various ions based on equilibrium relation with ion concentration in the external environment.

Further, in order to enhance ion sustained releasability of the ion sustained-release glass, it is preferable to functionalize the glass surface by surface treatment, thereby improving the ion sustained releasability. Specific examples of a surface treatment agent used in the surface treatment include one or more selected from surfactants, fatty acids, organic acids, inorganic acids, monomers, polymers, various coupling agents, silane compounds, metal alkoxide compounds, partially-condensed products thereof, and the like. Among these surface treatment agents, it is preferable to perform composite surface treatment using an acidic polymer and a silane compound.

The composite surface treatment is a method for surface treatment by using an acidic polymer after coating the surface of ion sustained-release glass with a silane compound. Specific examples are described below. In an aqueous dispersion containing an ion sustained-release glass finely ground to a desired average particle size by pulverization or the like, a silane compound is mixed, and this is hydrolyzed or partially hydrolyzed in the system to prepare a silanol compound. The silanol compound is then condensed to form polysiloxane, and then the surface of the ion sustained-release glass is coated with the polysiloxane to obtain a polysiloxane-coated ion sustained-release glass.

Specific examples of the silane compound that may be used for polysiloxane coating include one or more selected from tetramethoxysilane, tetraethoxysilane, tetrapropoxysilane, tetraallyloxysilane, tetrabutoxysilane, tetrakis(2-ethylhexyloxy) silane, trimethoxychlorosilane, triethoxychlorosilane, triisopropoxychlorosilane, trimethoxyhydroxysilane, diethoxydichlorosilane, tetraphenoxysilane, tetrachlorosilane, silicon hydroxide (silicon oxide hydrate), and the like. Tetramethoxysilane and/or tetraethoxysilane are more preferred.

More preferably, the silane compound to be used for polysiloxane coating is a low condensate. Examples include a low condensed silane compound obtained by partial hydrolysis and condensation of tetramethoxysilane and/or tetraethoxysilane. These compounds may be used singly or in combination.

The polysiloxane-coated ion sustained-release glass obtained in the above step may be subjected to acidic polymer treatment for a reaction with an acidic polymer to obtain an ion sustained-release glass. The acidic polymer treatment may be performed by using any equipment commonly used in the art as long as it is a dry fluidized bed stirrer. Examples of the equipment include Henschel mixer, super mixer, and high-speed mixer. The reaction of an acidic polymer with the ion sustained-release glass coated with polysiloxane may be performed by contacting an acidic polymer solution by impregnation, spraying, or the like. As an example, the polysiloxane-coated ion sustained-release glass may be dry-fluidized, and then the acidic polymer solution may be dispersed from above the fluidized bed and stirred well. The method for dispersing the acidic polymer solution is not particularly limited, but dropping or spraying is more preferred to achieve uniform dispersion. The reaction is preferably performed around room temperature. With higher temperatures, the reaction between an acid reactive element and the acidic polymer proceeds faster, resulting in a nonuniform cement phase.

It is preferable to remove water content in the cement reaction phase by performing heat treatment after the reaction. The water content remaining in the cement reaction phase is disadvantageous in terms of strength, but the polysiloxane coating suppresses reduction in mechanical strength of the filler used in the present embodiment. The heat treatment after the acidic polymer treatment is not limited to particular methods and may be performed by a known common method. Preferred equipment used for the heat treatment includes a box-type hot air dryer and a rotary heat treatment device capable of uniform heating. The heat treatment temperature is in the range from room temperature to 200° C. and more preferably in the range from 40 to 150° C. When the temperature is lower than this range, there is a risk that the removal of the aqueous medium is insufficient, and when the temperature is higher than this range, there is a risk that the organic layer of the acidic polymer is decomposed or discolored. The heat treatment duration depends on the capacity of a dryer and the like, and there is no problem as long as the aqueous medium is sufficiently removed. After the heat treatment, the heat-treated product may be easily deagglomerated by application of shear force or impact force, and the deagglomeration may be performed by, for example, the equipment used in the reaction.

The solvent used in preparing the acidic polymer solution for use in the reaction may be any solvent that dissolves the acidic polymer. Examples of the solvent include one or more selected from water, ethanol, isopropanol, acetone, and the like. Among these, water is particularly preferred. When water is used, the acidic group of the acidic polymer may dissociate and react uniformly with the surface of the basic filler which is the core.

The acidic polymer has a weight average molecular weight in the range of 2000 to 50000, and preferably in the range of 5000 to 40000. If the polysiloxane-coated ion sustained-release glass is treated with an acidic polymer having a weight average molecular weight of less than 2000, there is a tendency that an acidic polymer reaction phase is not formed in the polysiloxane-coated ion sustained-release glass, resulting in lower ion sustained releasability. On the other hand, if the polysiloxane-coated ion sustained-release glass is treated with an acidic polymer having a weight average molecular weight exceeding 50000, the viscosity of the acidic polymer solution becomes high, making it difficult to uniformly treat the polysiloxane-coated ion sustained-release glass. The concentration of the acidic polymer in 100 parts by mass of the acidic polymer solution is preferably in the range of 3 to 25 parts by mass, and more preferably in the range of 8 to 20 parts by mass. When the concentration of the acidic polymer is less than 3 parts by mass, the acidic polymer reaction phase is brittle, and therefore the effect of improving the sustained release of ions is not obtained. When the concentration of the acidic polymer exceeds 25 parts by mass, it is difficult to diffuse the polysiloxane layer (porous) in a uniform state, and a homogeneous acidic polymer reaction phase is not obtained. In addition, the reaction occurs immediately after contact with the polysiloxane-coated ion sustained-release glass, causing formation of strongly reacted agglomerates. The addition amount of the acidic polymer solution relative to the polysiloxane-coated ion sustained-release glass is preferably in the range of 6 to 40 parts by mass, and more preferably 10 to 30 parts by mass. When converted with this addition amount, the optimal amount of the acidic polymer relative to the polysiloxane-coated ion sustained-release glass is in the range of 1 to 7 parts by mass, and the optimal amount of water is in the range of 10 to 25 parts by mass.

The acidic polymer that may be used to form the acidic polymer reaction phase on the surface of the polysiloxane-coated ion sustained-release glass by the method described above may be any copolymer or homopolymer of polymerizable monomers having an acidic group such as phosphoric acid residue, pyrophosphoric acid residue, thiophosphoric acid residue, carboxylic acid residue, or sulfonic acid residue. Specific examples of these polymerizable monomers include one or more selected from acrylic acid, methacrylic acid, 2-chloroacrylic acid, 3-chloroacrylic acid, aconitic acid, mesaconic acid, maleic acid, itaconic acid, fumaric acid, glutaconic acid, citraconic acid, 4-(meth)acryloyloxyethoxycarbonyl phthalic acid, 4-(meth)acryloyloxyethoxycarbonyl phthalic anhydride, 5-(meth)acryloylaminopentyl carboxylic acid, 11-(meth)acryloyloxy-1,1-undecane dicarboxylic acid, 2-(meth)acryloyloxyethyl dihydrogen phosphate, 10-(meth)acryloyloxydecyl dihydrogen phosphate, 20-(meth)acryloyloxyeicosyl dihydrogen phosphate, 1,3-di(meth)acryloyloxypropyl-2-dihydrogen phosphate, 2-(meth)acryloyloxyethylphenyl phosphate, 2-(meth)acryloyloxyethyl-2′-bromoethyl phosphate, (meth)acryloyloxyethylphenyl phosphonate, di(2-(meth)acryloyloxyethyl) pyrophosphate, 2-(meth)acryloyloxyethyl dihydrogen dithiophosphate, 10-(meth)acryloyloxydecyl dihydrogen thiophosphate, and the like. Among the polymers obtained by (co) polymerization of these polymerizable monomers, a homopolymer or a copolymer of α-β unsaturated carboxylic acid which is relatively slow in acid-base reaction with an acid reactive element contained in the polysiloxane-coated ion sustained-release glass is preferred. Specific examples include one or more selected from acrylic acid polymers, acrylic acid-maleic acid copolymers, and acrylic acid-itaconic acid copolymers.

Filler (E) described above may be treated with a surface treatment agent such as a silane coupling agent to improve the affinity to the polymerizable monomer, the dispersibility in the polymerizable monomer, and the mechanical strength and water resistance of the cured product. The surface treatment agent and the surface treatment method are not particularly limited, and known methods, such as a method of spraying the surface treatment agent while stirring the powdery filler, a method of dispersing and mixing the filler and the surface treatment agent in a solvent, and a method of supplying a vapor or gaseous silane coupling agent to the filler surface, may be employed without limitation. As the silane coupling agent for use in the surface treatment of the filler, methyltrimethoxysilane, methyltriethoxysilane, methyltrichlorosilane, dimethyldichlorosilane, trimethylchlorosilane, vinyltrichlorosilane, vinyltriethoxysilane, vinyltris(2-methoxyethoxy) silane, 3-methacryloyloxypropyltrimethoxysilane, 3-chloropropyltrimethoxysilane, 3-glycidoxypropyltrimethoxysilane, 3-(meth)acryloxypropyltrimethoxysilane, 8-(meth)acryloxyoctyltrimethoxysilane, 11-(meth)acryloxyundecyltrimethoxysilane, hexamethyldisilazane, and the like are preferred. In addition to the silane coupling agent, the surface treatment of the filler may be performed by a method using one or more selected from titanate coupling agents, aluminate coupling agents, and the like. The treatment amount of the surface treatment agent in the filler is preferably 0.01 to 30 parts by mass, and more preferably 0.5 to 20 parts by mass, with respect to 100 parts by mass of the filler before treatment.

The shape of filler (E) is not particularly limited. The filler of any shape such as a spherical shape, a needle shape, a plate shape, a crushed shape, or a scale shape may be used. The average particle size of the filler is preferably 0.01 to 50 μm, more preferably 0.01 to 30 μm, even more preferably 0.05 to 20 μm, and particularly preferably 0.05 to 10 μm.

[(E1) Hydrophobized Silica Fine Particles with Primary Particle Size of Less Than 0.1 μm]

Dental photocurable composition (II) used in the present embodiment contains hydrophobized silica fine particles (E1) with a primary particle size of less than 0.1 μm (also referred to as “component (E1)”). Component (E1) is used as a viscosity modifier in the present embodiment. The use of component (E1) provides rheological properties and good operability that moderately prevents the dental photocurable composition from dripping. In addition, the development of good durable adhesive strength may be expected because the dental photocurable composition can be placed in an appropriate position. In the use of hydrophobized silica fine particles (E1) having a primary particle size of less than 0.1 μm as a viscosity modifier in the present embodiment, it is important that they are an inorganic filler. The use of hydrophobized silica fine particles (E1) is preferable because hydrophobized silica fine particles (E1) may improve mechanical strength and, in addition, they react with a polymerization initiator and the like contained in dental adhesive composition (I) and dental photocurable composition (II) used in the present embodiment and are less likely to cause reduction in curability.

Specific examples of a method for hydrophobizing silica fine particles include surface treatment with a modified silicone oil such as dimethylsilicone oil and/or surface treatment with a silane coupling agent having one or more functional groups selected from trimethylsilyl group, dimethylsilyl group, methylsilyl group, and alkylsilyl group optionally having a (meth)acryloyl group with an alkyl chain having 3 to 18 carbon atoms. It is preferable that the silane coupling agent hydrophobizes the silica fine particles via covalent bonding.

Specific examples of the modified silicone oil and the silane coupling agent include polydimethylsiloxane, hexamethyldisilazane, dimethylpolysiloxane, methylchlorosilane, alkylalkoxysilanes such as alkyltrialkoxysilane, dialkyldialkoxysilane, octadecylalkoxysilane, and octylalkoxysilane, and one or more (meth)acryloylalkylalkoxysilanes selected from 3-methacryloylpropyltrimethoxysilane, 8-methacryloyloctyltrimethoxysilane, and the like.

To further improve rheological properties, the silica fine particles may be hydrophobized by multiple methods. For example, after the treatment with a silane coupling agent and/or a modified silicone oil, or at the same time, the silica fine particles may be treated with a surface treatment agent.

Examples of hydrophobized silica fine particles (E1) with a primary particle size of less than 0.1 μm include dry silica, silica aerogel, wet silica, and the like. Dry silica is more preferred.

Among the hydrophobized silica fine particles listed above, dry silica is commercially available, for example, under the product name Aerosil from NIPPON AEROSIL CO., LTD. For example, silica fine powders with hydrophobic treatment on the surface may be used, such as Aerosil 50, Aerosil 90, Aerosil 130, Aerosil 200, Aerosil 300, Aerosil 380, Aerosil OX50, Aerosil TT600, Aerosil R972, Aerosil R974, Aerosil R976, Aerosil R976S, Aerosil R202, Aerosil R812, Aerosil R812S, Aerosil R805, Aerosil R104, Aerosil RI06, RY200, RX200, R711, RY200S, RA200H, R8200, and RA200HS.

The amount of hydrophobized silica fine particles (E1) with a primary particle size of less than 0.1 μm is 0.5 to 20 parts by mass with respect to 100 parts by mass of polymerizable monomer (A) contained in dental photocurable composition (II). When the amount of hydrophobized silica fine particles (E1) with a primary particle size of less than 0.1 μm is equal to or more than the above lower limit, dental photocurable composition (II) tends to exhibit rheological properties. When the amount of hydrophobized silica fine particles (E1) with a primary particle size of less than 0.1 μm is equal to or less than the above upper limit, the operability of dental photocurable composition (II) tends to be good.

The primary particle size in the present embodiment refers to the average primary particle size. The average particle size of filler (E) may be an average particle size calculated based on a particle size distribution measured by, for example, a laser diffraction particle size distribution analyzer. For example, the primary particle size may be measured using a laser diffraction particle size analyzer (Microtrac MT3300EXII available from NIKKISO CO., LTD). When using a laser diffraction particle size analyzer, the filler is dispersed by ultrasonic waves or the like to ensure uniformity before measurement. Alternatively, the primary particle size may be measured by dynamic light scattering particle size analysis, or by using electron micrographs for primary particles that are heavily agglomerated to form secondary particles. For a filler with a primary particle size of less than 0.1 μm, it is preferable to calculate the primary particle size from electron micrographs, and for a filler with a primary particle size of 0.1 μm or more, it is preferable to calculate the primary particle size by a laser diffraction particle size distribution analyzer.

It is preferable that dental photocurable composition (II) used in the present embodiment contains filler (E) in an amount of 10 to 400 parts by mass with respect to 100 parts by mass of polymerizable monomer (A) contained in dental photocurable composition (II). When filler (E) is present in an amount equal to or more than the above lower limit, good operability and durable adhesive strength tend to be exhibited. When filler (E) is present in an amount equal to or less than the above upper limit, the ease of use tends to be achieved with good paste properties.

Dental adhesive composition (I) used in the present embodiment is preferably free of filler (E). When dental adhesive composition (I) is sufficiently thick, the color tone stability is poor due to the inclusion of aromatic tertiary amine compound (D1). However, in actual use, discoloration, if it occurs, will not be visible because the film thickness is thin. The thinness of the film thickness is important because it also affects the durable adhesive strength, and in the present embodiment, the film thickness of dental adhesive composition (1) is usually 50 μm or less, preferably 20 μm or less, and more preferably 5 μm or less.

### < (F) Compound Containing Disulfide Group>

Dental adhesive composition (I) included in the present dental adhesive kit may contain a compound (F) containing a disulfide group (also referred to as “component (F)”). If compound (F) containing a disulfide group has a polymerizable group and corresponds to component (A), it is treated as component (F) in the present embodiment. This is because component (F) is copolymerizable due to the presence of the polymerizable group but the amount of component (F) is smaller than that of component (A).

Dental adhesive composition (I) has polymerizable monomer (A1) having an acidic group and aromatic tertiary amine compound (D1) and thus exhibits good durable adhesive strength to enamel. However, when polymerizable monomer (A1) having an acidic group and aromatic tertiary amine compound (D1) coexist, discoloration or gelation of dental adhesive composition (I) may occur. It has been confirmed that the discoloration and gelation may be suppressed when component (F) is blended in dental adhesive composition (I). It has also been confirmed that the suppression of discoloration and gelation by component (F) tends to be effective even in a small amount, and that the effect on durable adhesive strength is less than that of known stabilizers. The upper limit of the amount of component (F) is preferably 0.001 parts by mass or more and more preferably 0.01 parts by mass or more with respect to 100 parts by mass of dental adhesive composition (I). The amount of component (F) is preferably 0.001 to 2 parts by mass and more preferably 0.01 to 2 parts by mass with respect to 100 parts by mass of dental adhesive composition (I). When the amount of component (F) is equal to or more than the above lower limit, discoloration and gelation tend to be suppressed. When the amount of component (F) is equal to or less than the upper limit, good durable adhesive strength may be maintained.

Examples of compound (F) containing a disulfide group include one or more selected from 10-methacryloxydecyl-6,8-dithioctanate, 6-methacryloxyhexyl-6,8-dithioctanate, 2-methacryloyloxyethyl thioctate, thioctic acid (lipoic acid), N,N′-bis(acryloyl) cystamine, bis(2-hydroxyethyl)disulfide, difurfuryl disulfide, bis(3-carboxypropyl) disulfide, and the like. In addition to compounds (F) containing a disulfide group listed above, any known compound having a disulfide group (—S—S—) may be used. As an example of component (F) that may be preferably used, a compound having a structure derived from a thioctic group is preferred. Specific examples include one or more selected from 10-methacryloxydecyl-6,8-dithioctanate, 6-methacryloxyhexyl-6,8-dithioctanate, 2-methacryloyloxyethyl thioctate, thioctic acid (lipoic acid), and the like. When such a compound is used, discoloration and gelation may be suppressed with a smaller amount of the compound. Among these, thioctic acid is even more preferred. With a large amount of highly hydrophobic compound such as 10-methacryloxydecyl-6,8-dithioctanate, dental adhesive composition (I) containing water may become cloudy, or precipitates may form in the composition. On the other hand, thioctic acid (lipoic acid) has a low molecular weight and is less hydrophobic than 10-methacryloxydecyl-6,8-dithiocyanate. Therefore, the use of thioctic acid significantly reduces the possibility of precipitation in dental adhesive composition (I). Thioctic acid (lipoic acid) is an optical isomer, and either the R or L form may be used in the present embodiment. There is no problem even if thioctic acid (lipoic acid) partially contains β-lipoic acid which is the oxidized form, or dihydrolipoic acid which is the reduced form.

### <Other Components>

The dental photocurable composition used in the present embodiment may contain a component other than components (A) to (F) within a range that does not impair the effect of the present disclosure. For example, a UV absorber such as benzophenone and benzotriazole; a polymerization inhibitor such as hydroquinone, hydroquinone monomethyl ether, and 2,5-di-tert-butyl-4-methylphenol; a chain transfer agent such as α-alkylstyrene compound, mercaptan compound such as n-butyl mercaptan and n-octyl mercaptan, and terpenoid compound such as limonene, myrcene, α-terpinene, β-terpinene, γ-terpinene, terpinolene, β-pinene, and α-pinene; a metal capturing agent such as aminocarboxylic acid chelating agent and phosphonic acid chelating agent; a discoloration inhibitor; an antibacterial agent; a coloring pigment; a chemical polymerization initiator; and other additives conventionally known may be optionally added as necessary.

Dental adhesive composition (I) in the present dental adhesive kit may contain components (A), (B), (C), and (D) as essential components. In addition to these, dental adhesive composition (I) may contain one or more of the above optional components as necessary. In a certain aspect, dental adhesive composition (I) may include only components (A), (B), (C), and (D).

Dental photocurable composition (II) in the present dental adhesive kit may contain components (A), (D′), and (E) as essential components. In addition to these, dental photocurable composition (II) may contain one or more of the above optional components as necessary. In a certain aspect, dental photocurable composition (II) may include only components (A), (D′), and (E).

### [Chemical Polymerization Initiator]

The present dental adhesive kit may contain a chemical polymerization initiator (excluding components (D) and (D′) described above). The chemical polymerization is a polymerization method for curing without the need for special equipment such as a light irradiator. The chemical polymerization initiator is a polymerization initiator capable of initiating chemical polymerization. These are not particularly limited, and any known compounds commonly used may be used without any limitation.

As specific examples of a transition metal compound that may be used in the present dental adhesive kit, a copper (Cu) compound or a vanadium (V) compound may be preferably used. Examples of the copper (Cu) compound include one or more selected from copper chloride (monovalent), copper bromide (monovalent), copper chloride (divalent), copper acetate (divalent), copper gluconate (divalent), acetylacetone copper (divalent), copper methacrylate (divalent), and the like. Examples of the vanadium compound include one or more selected from acetylacetone vanadium (trivalent), divanadium tetraoxide (tetravalent), vanadyl acetylacetonate (tetravalent), vanadium oxide stearate (tetravalent), vanadyl oxalate (tetravalent), vanadyl sulfate (tetravalent), oxobis(1-phenyl-1,3-butanedioate) vanadium (tetravalent), bis(maltolate)oxovanadium (tetravalent), vanadium pentaoxide (pentavalent), sodium metavanadate (pentavalent), and the like.

Specific examples of a thiourea compound that may be used in the present dental adhesive kit include one or more selected from dimethylthiourea, diethylthiourea, tetramethylthiourea, (2-pyridyl)thiourea, N-methylthiourea, ethylenethiourea, N-allylthiourea, N-allyl-N′-(2-hydroxyethyl)thiourea, N-benzylthiourea, 1,3-dicyclohexylthiourea, N,N′-diphenylthiourea, 1,3-di(p-tolyl)thiourea, 1-methyl-3-phenylthiourea, N-acetylthiourea, N-benzoylthiourea, diphenylthiourea, dicyclohexylthiourea, and the like. Among these, one or more selected from (2-pyridyl)thiourea, N-acetylthiourea, N-benzoylthiourea, and N-benzylthiourea may be preferably used.

Examples of an organic peroxide that may be used in the present dental adhesive kit include one or more selected from diacyl peroxides, peroxyesters, dialkyl peroxides, peroxyketals, ketone peroxides, peroxyesters, peroxydicarbonates, and hydroperoxides. Among these, one or more selected from t-butylperoxy-2-ethylhexanoate, t-butylperoxybenzoate, t-amylperoxy-2-ethylhexanoate, t-amylperoxyacetate, t-amylperoxybenzoate, 1,1-di(t-butylperoxy)cyclohexane, 1,1-di(t-amylperoxy)cyclohexane, dibenzoyl peroxide, cumene hydroperoxide, t-butyl hydroperoxide, 1,1,3,3-tetramethylbutyl hydroperoxide, and the like may be used.

Other chemical polymerization initiators include one or more selected from phosphine compounds, sulfinic acid compounds, borate compounds, barbituric acid derivatives, and ascorbic acid compounds. Specific examples of the phosphine compounds include one or more phosphine compounds selected from triphenylphosphine and 4-(phenylphosphino)benzoic acid. Specific examples of the sulfinic acid compounds include one or more sulfinic acid compounds selected from sodium benzenesulfinate, sodium p-toluenesulfinate, and sodium 2,4,6-triisopropylbenzenesulfinate. The borate compounds include one or more borate compounds selected from sodium, lithium, potassium, and tetrabutylammonium salts of tetraarylborate compounds. The barbituric acid derivatives include one or more barbituric acid derivatives selected from 5-butylbarbituric acid, 1,3,5-trimethylbarbituric acid, 1-cyclohexyl-5-ethylbarbituric acid, and sodium or calcium salts of the above barbituric acid derivatives. Specific examples of the ascorbic acid compounds include one or more ascorbic acid compounds selected from ascorbic acid, ascorbyl 6-palmitate, and salt compounds of the above ascorbic acid compounds.

There is no problem if the polymerization initiator included in the present dental adhesive kit is subjected to secondary treatment such as fine grinding, adsorption on a carrier, and encapsulation in a microcapsule, as necessary. Furthermore, these polymerization initiators may be used singly or in combination of two or more, regardless of the polymerization manner or the polymerization method.

In a method for using the present dental adhesive kit, dental adhesive composition (I) is applied to an adherend and then air-dried as appropriate. The application surface of dental adhesive composition (I) may or may not be subjected to light irradiation. For the present dental adhesive kit, it is preferable that the application surface of dental adhesive composition (I) is not subjected to light irradiation. Light irradiation increases the number of operation steps, and the durable adhesive strength is better without light irradiation. In a common combination of a dental bonding agent and a dental composite resin, the application surface of the dental bonding agent is subjected to light irradiation. The present dental adhesive kit is different in this respect. The present dental adhesive kit is used in a procedure of laminating dental photocurable composition (II) on the application surface of dental adhesive composition (I), and applying light irradiation to dental photocurable composition (II) to cure dental photocurable composition (II).

When the present dental adhesive kit is used, a dental etching agent may be further used. The dental etching agent is a material containing an acidic compound and water, and one that contains phosphoric acid as the acidic compound is considered as a phosphoric acid etching agent. In particular, when bonding to unground enamel, a dental etching agent may be used in combination so that even higher adhesive strength may be exhibited. The dental etching agent is a composition containing an acidic component such as phosphoric acid, water, and a rheology modifier as appropriate, and is used to decalcify the enamel surface to form surface irregularities. In use in combination with the present dental adhesive kit, treatment with a dental etching agent, application of dental adhesive composition (I), and lamination of dental photocurable composition (II) may be performed in sequence. On the other hand, the use of a dental etching agent increases the number of operation steps.

The present dental adhesive kit may be used in a procedure of laminating dental photocurable composition (II) and applying light irradiation to dental photocurable composition (II), without light irradiation to the application surface of dental adhesive composition (I).

The present dental adhesive kit is suitable for bonding to tooth enamel and may be used, for example, for dental splinting procedure and also be applied in orthodontic treatment.

### EXAMPLES

The materials used in Examples and Comparative Examples and their abbreviations are listed below.

### <(A) Polymerizable Monomer>

### [(A1) Polymerizable Monomer Having Acidic Group]

MDP: 10-methacryloyloxydecyl dihydrogen phosphate
MET: 4-methacryloxyethyltrimellitic acid
META: 4-methacryloyloxyethoxycarbonylphthalic anhydride

### [(A2) Polymerizable Monomer Having No Acidic Group]

BisGMA: 2,2-bis[4-(3-methacryloyloxy-2-hydroxypropoxy)phenyl]propane
UDMA: N,N-(2,2,4-trimethylhexamethylene)bis[2-(aminocarboxy) ethanol]methacrylate
TEGDMA: triethylene glycol dimethacrylate
GDMA: glycerol dimethacrylate
HEMA: hydroxyethyl methacrylate
MPTMS: (3-methacryloyloxypropyl) trimethoxysilane

### <(B) Water>

DW: distilled water

### <(C) Volatile Organic Solvent>

EtOH: ethanol
Ac: acetone

### <(D) or (D′) Photopolymerization Initiator>

### [(D1) Aromatic Tertiary Amine Compound]

DMBE: ethyl dimethylaminobenzoate
DMBN: 4-(dimethylamino)benzonitrile
DEPT: N,N-di(2-hydroxyethyl)-p-toluidine

### [(D2) α-Diketone Compound]

CQ: camphorquinone

### [(D3) Iodonium Salt Compound]

TBIB: bis(p-tert-butylphenyl) iodonium tetrakis(pentafluorophenyl) borate
CTIP: p-cumenyl (p-tolyl) iodonium
tris(pentafluoroethyl)trifluorophosphate
TBIG: bis(p-tert-butylphenyl) iodonium tetrakis(pentafluorophenyl) gallate

### [(D4) Aliphatic Tertiary Amine Compound Having Aromatic Ring]

TBA: tribenzylamine
DBMA: N-benzyl-N-methylbenzylamine
DBAE: N,N-dibenzylglycine ethyl ester

### [Other Photopolymerization Initiator]

DMAEMA: dimethylaminoethyl methacrylate
MDEA: N-methyldiethanolamine

### <(E) Filler>

### [Filler E-1]

To 100 g of a zirconium silicate filler (average particle size 1.5 μm, 20% by mass of zirconia, 80% by mass of silica), a silane coupling treatment solution containing 20 g of water, 35 g of ethanol, and 3 g of 3-methacryloyloxypropyltrimethoxysilane as a silane coupling agent was added and mixed by stirring for 2 hours. The mixture was then heat-treated at 90° C. for 15 hours and then sieved to obtain filler E-1.

### [Filler E-2]

To 100 g of a barium silicate filler (average particle size 1 μm, 10% by mass of Al₂O₃, 10% by mass of B₂O₃, 25% by mass of BaO, 55% by mass of SiO₂), a silane coupling treatment solution containing 20 g of water, 35 g of ethanol, and 6 g of 3-methacryloyloxypropyltrimethoxysilane as a silane coupling agent was added and mixed by stirring for 2 hours. The mixture was then heat-treated at 90° C. for 15 hours and then sieved to obtain filler E-2.

### [Filler E-3]

To 100 g of fluoroaluminoborosilicate glass (average particle size 1 μm, 22.5% by mass of SiO₂, 20.0% by mass of Al₂O₃, 12.3% by mass of B₂O₃, 35.7% by mass of SrO, 2.5% by mass of Na₂O, 7.0% by mass of F), a silane coupling treatment solution containing 20 g of water, 35 g of ethanol, and 6 g of 3-methacryloyloxypropyltrimethoxysilane as a silane coupling agent was added and mixed by stirring for 2 hours. The mixture was then heat-treated at 90° C. for 15 hours and then sieved to obtain filler E-3.

### [Filler E-4]

To 100 g of fluoroaluminoborosilicate glass (average particle size 1 μm, 22.5% by mass of SiO₂, 20.0% by mass of Al₂O₃, 12.3% by mass of B₂O₃, 35.7% by mass of SrO, 2.5% by mass of Na₂O, 7.0% by mass of F), 4.5 g of a low condensate of silane compound “MKC Silicate MS56S” (SiO₂ content 56.0% by mass, degree of polymerization 2 to 100, manufactured by Mitsubishi Chemical Corporation) was added and mixed by stirring for about 90 minutes. After mixing for a predetermined period of time, the resulting treated slurry was aged in a hot air dryer at 50° C. for 40 hours, then heated to 150° C. and held for 6 hours, and then cooled to obtain a heat-treated product. The obtained heat-treated product was placed in a Henschel mixer and deagglomerated at 1800 rpm for 5 minutes. Then, 16.0 g of an aqueous polyacrylic acid solution (polymer concentration 13% by mass, weight average molecular weight 10,000, manufactured by NACALAI TESQUE, INC.) was sprayed from above. After spraying, the powder taken out from the mixer was heated in a hot air dryer at 100° C. for 3 hours. To 100 g of the produced filler, a silane coupling treatment solution prepared by stirring 100 g of water, 80 g of ethanol, and 9 g of 3-methacryloyloxypropyltrimethoxysilane as a silane coupling agent at room temperature for 2 hours was added and mixed by stirring for 30 minutes. The mixture was then heat-treated at 90° C. for 15 hours to obtain filler E-4.

### [Filler E-5]

A uniform matrix was prepared by mixing 60 parts by mass of BisGMA, 40 parts by mass of TEGDMA, and 0.2 parts by mass of BPO. The prepared matrix, 300 parts by mass of fluoroboroaluminosilicate glass (average primary particle size 0.5 μm), 15 parts by mass of Aerosil R711, and 15 parts by mass of γ-methacryloxypropyltrimethoxysilane were mixed and polymerized in a nitrogen atmosphere using a kneader to obtain a cured product. The cured product was coarsely ground using a roll crusher and then finely ground using a vibration mill to obtain a finely ground product having an average particle size of 20 μm. Then, 100 parts by mass of the sieved finely ground product, 1 part by mass of water, 1 part by mass of ethanol, and 6 parts by mass of γ-methacryloxypropyltrimethoxysilane were mixed, and the mixture was dried at 90° C. for 10 hours to obtain filler E-5, which was an organic-inorganic composite filler.

### [Filler E-6]

Titanium oxide (average particle size 0.25 μm)
   [(E1) Hydrophobized Silica Fine Particles with Primary Particle Size of Less Than 0.1 μm]

### (Filler E1-1)

Aerosil R-8200 (manufactured by Evonik Industries AG)

### (Filler E1-2)

Aerosil R-711 (manufactured by Evonik Industries AG)

### <(F) Compound Containing Disulfide Group>

MDDT: 10-methacryloxydecyl-6,8-dithioctanate
MEDT: 2-methacryloyloxyethyl thioctate
THAC: thioctic acid

### <Others>

### [Polymerization Inhibitor]

MeHQ: p-methoxyphenol
BHT: dibutylhydroxytoluene

### [UV Absorber]

OB: 2-hydroxy-4-(octyloxy)benzophenone

### [Fluorescent Agent]

FA: diethyl 2,5-dihydroxyterephthalate

### Production Example 1-1: Method for Producing Dental Adhesive Composition (I) (1-1)

All components listed in Table 1 were put into a wide-mouth plastic container and mixed by using Mixed Rotor VMRC-5 at 100 rpm for 48 hours to obtain dental adhesive composition (I) (1-1). In Table 1, the content (parts by mass) of each component is listed in parentheses after the abbreviation of the component.

### Production Examples 1-2 to 1-11 and Comparative Production Examples 1-1 to 1-4

Production Examples 1-2 to 1-11 and Comparative Production Examples 1-1 to 1-4 were obtained by the same method as in Production Example 1-1, except that the compositions were changed to those listed in Tables 1 and 2. The numbers of Production Examples correspond to the numbers of dental adhesive compositions (I).

### Production Example 2-1: Method for Producing Dental Photocurable Composition (II) (2-1)

All components listed in Table 3 except filler (E) were put into a wide-mouth plastic container and mixed by using Mixed Rotor VMRC-5 at 100 rpm for 48 hours to obtain a matrix. The matrix and filler (E) were then fed into a kneading machine, stirred uniformly, and defoamed under vacuum to obtain dental photocurable composition (II) (2-1). In Table 3, the content (parts by mass) of each component is listed in parentheses after the abbreviation of the component.

### Production Examples 2-2 to 2-15 and Comparative Production Examples 2-1 to 2-4

Production Examples 2-2 to 2-15 and Comparative Production Examples 2-1 to 2-4 were obtained by the same method as in Production Example 2-1, except that the compositions were changed to those listed in Tables 3 and 4. The numbers of Production Examples correspond to the numbers of dental curable compositions.

### <Evaluation 1: Durable Adhesive Strength>

Test specimens of cow central incisors embedded in an epoxy resin were polished with #500 water-resistant abrasive paper and then polished with #1000 water-resistant abrasive paper to shave the enamel planes. Then, dental adhesive compositions (I) listed in Examples were applied to the adherend surfaces and air-dried. Only in Example 21, after the application surface of dental adhesive composition (I) was air-dried, light irradiation was performed. In Example 23, a dental etching agent (Opal Etch from Ultradent Products Inc.) was applied to the enamel surface, rinsed, and air-dried, and then dental adhesive composition (I) was applied, air-dried, and subjected to light irradiation. The light irradiation was performed with an LED light irradiator for dental polymerization (PEN Bright from Shofu Inc.) for 5 seconds. Next, a mold with a hole 2.38 mm in diameter (manufactured by Ultradent Products Inc.) was placed on top of the application surface of dental adhesive composition (I), and dental photocurable composition (II) listed in Examples was laminated and then subjected to light irradiation for 10 seconds using an LED light irradiator for dental polymerization (PEN Bright from Shofu Inc.). After removing the mold, the prepared adhesive test specimen was immersed in 37° C. water for 24 hours and then immersed in a cold water phase at 4° C. and a hot phase at 60° C. for 30 seconds each in a thermal shock tester (manufactured by Thomas Kagaku Co., Ltd.) for 5,000 cycles. The prepared test specimen was then subjected to a shear test in a universal testing system (manufactured by Instron) at a crosshead speed of 1 mm/min. A test result of 20 MPa or more was defined as extremely good durable adhesive strength. A test result of 15 MPa or more and less than 20 MPa was determined as normal durable adhesive strength, and a test result of less than 15 MPa was determined as insufficient durable adhesive strength.

### <Evaluation 2: Color Tone Stability>

Dental photocurable composition (II) was filled into a stainless steel mold (disk shape 15 mm in diameter×1 mm) and then pressed using a cover glass placed on the top. Curing was performed by light irradiation for 1 minute using an LED light irradiator for dental polymerization (PEN Bright from Shofu Inc.) from above the cover glass. The cover glass was removed, and then the cured product was removed from the mold. One side of the cured product of dental photocurable composition (II) was polished with #500 water-resistant abrasive paper, and then dental adhesive composition (I) was applied to the surface of the cured product of dental photocurable composition (II) and air-dried. Only in Example 21, after the application surface of dental adhesive composition (I) was air-dried, light irradiation was performed. The light irradiation was performed with an LED light irradiator for dental polymerization (PEN Bright from Shofu Inc.) for 5 seconds. Color measurement was performed for the surface of the cured product of dental photocurable composition (II) that was not coated with dental adhesive composition (I). Color measurement was performed by placing a test specimen on a standard white plate (D65/10° X=81.07, Y=86.15, Z=93.38) background and using a spectrocolorimeter (manufactured by BYK Chemie) under predetermined conditions (light source: C, viewing angle: 2°, measurement area: 11 mm). Then, the surface of the cured product of dental photocurable composition (II) that was not coated with dental adhesive composition (I) was exposed to light for 24 hours in a xenon lamp light exposure tester (SUNTEST CPS+), and the color tone of the test specimen was measured again. The difference of discoloration was expressed as ΔE calculated from the following formulae.

$$Δ E = {\{ {( Δ {L}^{⋆} )}^{2} + {( Δ {a}^{⋆} )}^{2} + {( Δ {b}^{⋆} )}^{2} \}}^{1 / 2}$$

$$Δ {L}^{⋆} = L {1}^{⋆} - L {2}^{⋆}$$

$$Δ {a}^{⋆} = a {1}^{⋆} - a {2}^{⋆}$$

$$Δ {b}^{⋆} = b {1}^{⋆} - b {2}^{⋆}$$

where L1* is the lightness index before light exposure, L2* is the lightness index after light exposure, a1* and b1* are the chromaticness index before light exposure, and a2* and b2* are the chromaticness index after light exposure. Those with ΔE of less than 9 were rated “A” as best, and those with ΔE of 9 or more were rated “C” as undesirable. When the color tone stability is good, high esthetic properties may be maintained with less discoloration during use.

### <Evaluation 3: Determination of Stability of Dental Adhesive Composition (I)>

The prepared dental adhesive composition (I) was filled in an amount of 5 mL into a polypropylene bottle and sealed by capping. The color tone of dental adhesive composition (I) was visually checked before storage in a constant temperature chamber, and then stored in a constant temperature chamber set at 50° C. for 2 months. After storage, dental adhesive composition (I) was drained from the bottle, and the color tone was visually checked before and after storage in the constant temperature chamber set at 50° C. Those with no change or a slight change in color tone were rated “A” as acceptable, and those with a significant change in color tone were rated “B” as undesirable. A significant change in color tone of dental adhesive composition (I) is undesirable because it causes discomfort to the operator.

The results of each test listed in Tables 5 and 6 are discussed.

TABLE 1
(I) Dental adhesive composition

| | Component (A) | | | | Component (D) | | | | | | |
|---|---|---|---|---|---|---|---|---|---|---|---|
| | Compo- | Compo- | Compo- | Compo- | Compo- | Compo- | Compo- | | Compo- | Compo- | |
| | nent | nent | nent | nent | nent | nent | nent | | nent | nent | |
| | (A1) | (A2) | (B) | (C) | (D1) | (D2) | (D4) | Others | (E) | (F) | Others |
| Production | MDP (2) | BisGMA (15) | D.W. | Acetone | DMBE (0.1) | CQ | — | — | — | MDDT | BHT |
| Example | META (8.5) | TEGDMA (5) | (25) | (44.4) | | (0.3) | | | | (0.1) | (0.1) |
| 1-1 | | MPTMS (0.5) | | | | | | | | | |
| Production | MHPA (3) | BisGMA (5) | D.W. | Acetone | DMBE (0.5) | CQ | — | — | — | MDDT | BHT |
| Example | MET (5) | | (31) | (54.6) | | (0.5) | | | | (0.3) | (0.1) |
| 1-2 | | | | | | | | | | | |
| Production | MDP (2) | BisGMA (15) | D.W. | Acetone | DMBE (0.6) | CQ | — | — | — | MDDT | BHT |
| Example | META (8) | GDMA (5) | (37.9) | (30) | | (0.3) | | | | (0.1) | (0.1) |
| 1-3 | | MPTMS (0.5) | | | | | | | | | |
| Production | MDP (2) | BisGMA (15) | D.W. | Acetone | DMBE (0.1) | CQ | — | — | — | THAC | BHT |
| Example | META (8.5) | TEGDMA (5) | (24.1) | (44.4) | | (0.29) | | | | (0.01) | (0.1) |
| 1-4 | | MPTMS (0.5) | | | | | | | | | |
| Production | MDP (15) | HEMA (10) | D.W. | EtOH | DMBE (0.6) | CQ | — | — | — | MEDT | BHT |
| Example | | BisGMA (13) | (14.9) | (40.9) | | (0.4) | | | | (0.1) | (0.1) |
| 1-5 | | GDMA (5) | | | | | | | | | |
| Production | MDP (4) | HEMA (15) | D.W. | Acetone | DMBN (0.6) | — | — | — | — | THAC | BHT |
| Example | META (10) | BisGMA (15) | (24.7) | (30.3) | | | | | | (0.3) | (0.1) |
| 1-6 | | | | | | | | | | | |
| Production | MDP (2) | — | D.W. | EtOH | DMBE (1.0) | — | — | — | — | THAC | — |
| Example | META (15) | | (35.4) | (43.8) | | | | | | (2.4) | |
| 1-7 | | | | | | | | | | | |

TABLE 2
(I) Dental adhesive composition

| | Component (A) | | | | Component (D) | | | | | | |
|---|---|---|---|---|---|---|---|---|---|---|---|
| | Compo- | Compo- | Compo- | Compo- | Compo- | Compo- | Compo- | | Compo- | Compo- | |
| | nent | nent | nent | nent | nent | nent | nent | | nent | nent | |
| | (A1) | (A2) | (B) | (C) | (D1) | (D2) | (D4) | Others | (E) | (F) | Others |
| Production | MDP (2) | BisGMA (15) | D.W. | Acetone | DMBE (0.2) | CQ | — | — | — | — | BHT |
| Example | META (8.5) | TEGDMA (5) | (25) | (44.4) | | (0.3) | | | | | (0.2) |
| 1-8 | | MPTMS (0.5) | | | | | | | | | |
| Production | MDP (5) | BisGMA (12.5) | D.W. | Acetone | DMBE (0.3) | — | — | — | E1-1 | THAC | BHT |
| Example | META (5) | HEMA (12.5) | (15.4) | (23) | | | | | (20) | (0.2) | (0.1) |
| 1-9 | | GDMA (6) | | | | | | | | | |
| | | [symbol] | | | | | | | | | |
| Production | MDP (2) | — | D.W. | Acetone | DMBE (0.3) | — | — | — | — | — | BHT |
| Example | | | (50) | (47.6) | | | | | | | (0.1) |
| 1-10 | | | | | | | | | | | |
| Production | MDP (5) | HEMA (17) | D.W. | Acetone | DMBE (0.2) | — | DBMA | — | — | — | BHT |
| Example | | | (16.7) | (60) | | | (2.0) | | | | (0.1) |
| 1-11 | | | | | | | | | | | |
| Production | MDP (2) | — | D.W. | EtOH | DMBE (1.0) | — | — | — | — | MDDT | — |
| Example | META (15) | | (35.6) | (44.3) | | | | | | (2.2) | |
| 1-12 | | | | | | | | | | | |
| Comparative | MHPA (3) | BisGMA (12) | D.W. | Acetone | — | — | — | DMAEMA | — | — | BHT |
| Production | MET (5) | HEMA (15) | (17.2) | (46.7) | | | | (1.0) | | | (0.1) |
| Example | | | | | | | | | | | |
| 1-1 | | | | | | | | | | | |
| Comparative | MDP (5) | HEMA (20) | D.W. | EtOH | — | — | — | — | — | — | BHT |
| Production | | | (21.7) | (53.2) | | | | | | | (0.1) |
| Example | | | | | | | | | | | |
| 1-2 | | | | | | | | | | | |
| Comparative | MDP (5) | — | — | Acetone | DMBE (0.2) | — | — | — | — | THAC | BHT |
| Production | | | | (94.3) | | | | | | (0.4) | (0.1) |
| Example | | | | | | | | | | | |
| 1-3 | | | | | | | | | | | |

[symbol]

indicates data missing or illegible when filed

TABLE 3
(II) Dental photocurable composition

| | Component (A) | | Component (D′) | | | | | Component (E) | | |
|---|---|---|---|---|---|---|---|---|---|---|
| | Compo- | Compo- | Compo- | Compo- | Compo- | Compo- | | | Compo- | |
| | nent | nent | nent | nent | nent | nent | | | nent | |
| | (A) | (A2) | (D1) | (D2) | (D3) | (D4) | Others | Others | (E1) | Others |
| Production | — | BisGMA(60) | — | CQ | TBIB | TBA | — | E-1 (130) | E1-1 | MeHQ (0.01) |
| Example | | TEGDMA(40) | | (0.10) | (2.5) | (2.5) | | | (15) | |
| 2-1 | | | | | | | | | | |
| Production | — | BisGMA(60) | — | CQ | TBIB | TBA | — | E-1 (130) | — | MeHQ (0.01) |
| Example | | TEGDMA(40) | | (0.10) | (2.5) | (2.5) | | | | |
| 2-2 | | | | | | | | | | |
| Production | MDP | UDMA(70) | — | CQ | CTIP | DBMA | — | E-2 (200) | E1-1 | OB (0.2) |
| Example | (3.0) | TEGDMA(27) | | (0.10) | (2.0) | (1.5) | | | (10) | MeHQ (0.01) |
| 2-3 | | | | | | | | | | FA (0.005) |
| Production | — | BisGMA(60) | — | CQ | TBIG | TBA | — | E-1 (130) | E1-1 | OB (0.2) |
| Example | | TEGDMA(40) | | (0.04) | (5.1) | (5.1) | | E-3 (50) | (12) | MeHQ (0.01) |
| 2-4 | | | | | | | | | | FA (0.005) |
| Production | — | UDMA(70) | — | CQ | TBIB | DBMA | — | E-1 (200) | E1-1 | OB (0.2) |
| Example | | TEGDMA(30) | | (0.6) | (0.3) | (0.5) | | | (10) | MeHQ (0.01) |
| 2-5 | | | | | | | | | | FA (0.005) |
| Production | — | BisGMA(60) | — | CQ | CTIP | DBMA | — | E-2 (200) | E1-1 | OB (0.2) |
| Example | | TEGDMA(40) | | (0.10) | (2.5) | (1.5) | | | (7) | MeHQ (0.01) |
| 2-6 | | | | | | | | | | FA (0.005) |
| Production | — | UDMA(70) | — | CQ | TBIG | TBA | — | E-1 (130) | E1-1 | MeHQ (0.01) |
| Example | | TEGDMA(30) | | (0.15) | (2.5) | (2.5) | | E-4 (50) | (15) | FA (0.005) |
| 2-7 | | | | | | | | | | |
| Production | — | BisGMA(60) | — | CQ | TBIB | TBA | — | E-3 (130) | E1-1 | OB (0.2) |
| Example | | TEGDMA(40) | | (0.30) | (2.5) | (2.5) | | E-4 (50) | (10) | MeHQ (0.01) |
| 2-8 | | | | | | | | | | FA (0.005) |
| Production | — | UDMA(70) | — | CQ | TBIB | TBA | — | E-3 (125) | E1-2 | OB (0.2) |
| Example | | TEGDMA(30) | | (0.10) | (2.5) | (2.5) | | E-4 (25) | (0. 5) | MeHQ (0.01) |
| 2-9 | | | | | | | | E-5 (250) | | FA (0.005) |
| Production | — | UDMA(70) | — | CQ | CTIP | TBA | — | E-3 (130) | E1-2 | MeHQ (0.01) |
| Example | | TEGDMA(30) | | (0.15) | (2.5) | (2.5) | | E-4 (50) | (15) | FA (0.005) |
| 2-10 | | | | | | | | | | |

TABLE 4
(II) Dental photocurable composition

| | Component (A) | | Component (D′) | | | | | Component (E) | | |
|---|---|---|---|---|---|---|---|---|---|---|
| | Compo- | Compo- | Compo- | Compo- | Compo- | Compo- | | | Compo- | |
| | nent | nent | nent | nent | nent | nent | | | nent | |
| | (A1) | (A2) | (D1) | (D2) | (D3) | (D4) | Others | Others | (E1) | Others |
| Production | — | BisGMA(60) | — | CQ | TBIG | TBA | — | E-3 (200) | E1-2 | OB (0.2) |
| Example | | TEGDMA(40) | | (0.10) | (2.5) | (2.5) | | E-6 (5) | (15) | MeHQ (0.01) |
| 2-11 | | | | | | | | | | FA (0.005) |
| Production | — | BisGMA(60) | — | CQ | TBIG | TBA | — | E-3 (200) | E1-2 | OB (0.2) |
| Example | | TEGDMA(40) | | (0.10) | (2.5) | (2.5) | | E-6 (5) | (15) | MeHQ (0.01) |
| 2-12 | | | | | | | | | | FA (0.005) |
| Production | — | BisGMA(35) | — | CQ | TBIB | DBMA | — | — | E1-2 | OB (0.2) |
| Example | | UDMA (35) | | (0.30) | (1.5) | (1.0) | | | (20) | MeHQ (0.01) |
| 2-13 | | TEGDMA(30) | | | | | | | | FA (0.005) |
| Production | MHPA (2) | BisGMA(35) | — | CQ | TBIG | TBA | — | — | E1-2 | MeHQ (0.01) |
| Example | META (2) | UDMA (35) | | (0.10) | (2.0) | (2.0) | | | (15) | |
| 2-14 | | TEGDMA(26) | | | | | | | | |
| Production | MHPA (2) | BisGMA(35) | — | CQ | TBIG | TBA | — | E-4 (10) | E1-2 | MeHQ (0.01) |
| Example | META (2) | UDMA (35) | | (0.10) | (1.0) | (1.5) | | | (15) | |
| 2-15 | | TEGDMA(26) | | | | | | | | |
| Comparative | MHPA (2) | BisGMA(60) | DMBE (1.0) | CQ | TBIG | — | DMAEMA | E-1 (130) | E1-2 | OB (0.2) |
| Production | META (2) | TEGDMA(40) | | (0.15) | (1.0) | | (1.0) | E-4 (50) | (15) | MeHQ (0.01) |
| Example | | | | | | | | | | FA (0.005) |
| 2-1 | | | | | | | | | | |
| Comparative | — | BisGMA(60) | DMBE (1.0) | CQ | TBIG | TBA | — | E-1 (130) | E1-2 | MeHQ (0.01) |
| Production | | TEGDMA(40) | | (0.10) | (1.0) | (1.0) | | E-4 (50) | (15) | FA (0.005) |
| Example | | | | | | | | | | |
| 2-2 | | | | | | | | | | |
| Comparative | — | BisGMA(60) | DMBE (1.0) | CQ | TBIG | — | — | E-3 (130) | E1-2 | MeHQ (0.01) |
| Production | | TEGDMA(40) | DEPT (0.2) | (0.30) | (1.0) | | | E-4 (50) | (15) | FA (0.005) |
| Example | | | | | | | | | | |
| 2-3 | | | | | | | | | | |
| Comparative | MHPA (2) | BisGMA(35) | — | CQ | TBIG | — | MDEA | — | E1-2 | OB (0.2) |
| Production | META (2) | UDMA (35) | | (0.10) | (1.0) | | (1.0) | | (15) | MeHQ (0.01) |
| Example | | TEGDMA(26) | | | | | | | | FA (0.005) |
| 2-4 | | | | | | | | | | |

TABLE 5

| | | | | | (I) Color tone |
|---|---|---|---|---|---|
| | (I) Dental | (II) Dental | Durable | | stability of |
| | adhesive | photocurable | adhesive | Color tone | Dental adhesive |
| | composition | composition | strength | stability | composition |
| Example 1 | Production | Production | 24.5 | A | A |
| | Example | Example | | | |
| | 1-2 | 2-1 | | | |
| Example 2 | Production | Production | 22.2 | A | A |
| | Example | Example | | | |
| | 1-1 | 2-2 | | | |
| Example 3 | Production | Production | 24.7 | A | A |
| | Example | Example | | | |
| | 1-2 | 2-3 | | | |
| Example 4 | Production | Production | 15.7 | A | A |
| | Example | Example | | | |
| | 1-3 | 2-4 | | | |
| Example 5 | Production | Production | 16.4 | A | A |
| | Example | Example | | | |
| | 1-4 | 2-5 | | | |
| Example 6 | Production | Production | 21.2 | A | A |
| | Example | Example | | | |
| | 1-2 | 2-6 | | | |
| Example 7 | Production | Production | 20.2 | A | A |
| | Example | Example | | | |
| | 1-5 | 2-7 | | | |
| Example 8 | Production | Production | 20.6 | A | A |
| | Example | Example | | | |
| | 1-6 | 2-8 | | | |
| Example 9 | Production | Production | 19.7 | A | A |
| | Example | Example | | | |
| | 1-7 | 2-3 | | | |
| Example 10 | Production | Production | 23.1 | A | B |
| | Example | Example | | | |
| | 1-8 | 2-1 | | | |
| Example 11 | Production | Production | 27.9 | A | A |
| | Example | Example | | | |
| | 1-1 | 2-9 | | | |
| Example 12 | Production | Production | 23.2 | A | A |
| | Example | Example | | | |
| | 1-1 | 2-10 | | | |
| Example 13 | Production | Production | 23.5 | A | A |
| | Example | Example | | | |
| | 1-1 | 2-11 | | | |
| Example 14 | Production | Production | 23.1 | A | A |
| | Example | Example | | | |
| | 1-1 | 2-12 | | | |

TABLE 6

| | | | | | (I) Color tone |
|---|---|---|---|---|---|
| | (I) Dental | (II) Dental | Durable | | stability of |
| | adhesive | photocurable | adhesive | Color tone | Dental adhesive |
| | composition | composition | strength | stability | composition |
| Example 15 | Production | Production | 20.1 | A | A |
| | Example | Example | | | |
| | 1-1 | 2-13 | | | |
| Example 16 | Production | Production | 20.6 | A | A |
| | Example | Example | | | |
| | 1-1 | 2-14 | | | |
| Example 17 | Production | Production | 20.1 | A | A |
| | Example | Example | | | |
| | 1-1 | 2-15 | | | |
| Example 18 | Production | Production | 16.0 | A | A |
| | Example | Example | | | |
| | 1-9 | 2-15 | | | |
| Example 19 | Production | Production | 17.1 | A | B |
| | Example | Example | | | |
| | 1-10 | 2-15 | | | |
| Example 20 | Production | Production | 17.4 | A | B |
| | Example | Example | | | |
| | 1-11 | 2-15 | | | |
| Example 21 | Production | Production | 16.3 | A | A |
| | Example | Example | | | |
| | 1-2 | 2-1 | | | |
| Example 22 | Production | Production | 17.2 | A | A |
| | Example | Example | | | |
| | 1-12 | 2-1 | | | |
| Example 23 | Production | Production | 30.6 | A | A |
| | Example | Example | | | |
| | 1-2 | 2-1 | | | |
| Comparative | Production | Comparative | 16.8 | C | A |
| Example 1 | Example | Production | | | |
| | 1-1 | Example | | | |
| | | 2-1 | | | |
| Comparative | Production | Comparative | 21.5 | C | A |
| Example 2 | Example | Production | | | |
| | 1-1 | Example | | | |
| | | 2-2 | | | |
| Comparative | Production | Comparative | 20.8 | C | A |
| Example 3 | Example | Production | | | |
| | 1-5 | Example | | | |
| | | 2-3 | | | |
| Comparative | Production | Comparative | 14.1 | A | A |
| Example 4 | Example | Production | | | |
| | 1-10 | Example | | | |
| | | 2-4 | | | |
| Comparative | Comparative | Production | 9.3 | C | A |
| Example 5 | Production | Example | | | |
| | Example | 2-1 | | | |
| | 1-1 | | | | |
| Comparative | Comparative | Production | 12.9 | A | A |
| Example 6 | Production | Example | | | |
| | Example | 2-13 | | | |
| | 1-2 | | | | |
| Comparative | Comparative | Production | 13.1 | A | A |
| Example 7 | Production | Example | | | |
| | Example | 2-13 | | | |
| | 1-3 | | | | |

The compositions listed in Examples were found to have both good durable adhesiveness to enamel and color tone stability.

In Examples 4 and 5, the durable adhesive strength tended to be slightly inferior. This may be because the amount of component (D2) is small in Production Example 2-4 and the amount of component (D3) is small in Production Example 2-5.

In Examples 18, 19, and 20, the durable adhesive strength tended to be slightly inferior. This may be because Production Example 1-9 includes component (E), Production Example 1-10 has a low content of component (A1), which is less than 3 parts by mass, and Production Example 1-11 includes aliphatic tertiary amine compound (D4) having an aromatic ring.

In Examples 10, 19, and 20, the color tone stability of dental adhesive composition (I) was poor. This may be because Production Example 1-8, Production Example 1-10, and Production Example 1-11 do not include component (F).

In Example 21, dental adhesive composition (I) was applied and then subjected to light irradiation, and thereafter dental photocurable composition (II) was applied. The durable adhesive strength tended to be lower than that of Example 1 in which light irradiation was not performed after dental adhesive composition (I) was applied.

In Example 22, the durable adhesive strength tended to be low. This may be because Production Example 1-12 was white and cloudy, and the cause of the cloudiness may be attributable to a large amount of MDDT blended. THAC is preferred among components (F) because of its high solubility in dental adhesive composition (I).

Although Example 23 exhibited the best durable adhesive strength among Examples performed this time, the operation is more complicated than that of the other Examples because of the treatment using a dental etching agent on enamel.

In Comparative Examples 1 to 3, the color tone stability tended to be poor. This may be because Comparative Production Examples 2-1 to 2-3 include component (D1). In Comparative Examples 4 to 7, the durable adhesive strength tended to be poor. In Comparative Example 4, this may be because Comparative Production Example 2-4 does not include components (D1) and (D4), and in combination with dental adhesive composition (I) with a small amount of component (A1) as in Production Example 1-10, sufficient adhesive strength may fail to be exhibited. In Comparative Examples 5 and 6, this may be because Comparative Examples 1-1 and 1-2 do not include component (D1). In Comparative Example 7, this may be because Comparative Production Example 1-3 does not include component (B).

The dental adhesive kits evaluated in Examples may be used for composite resins, resin cements, dental core build-up materials, dental orthodontic materials, dental sealant materials, dental splinting materials, and the like. Among these, the dental adhesive kits may be suitable for dental orthodontic materials, dental sealant materials, and dental splinting materials because high durable adhesive strength to enamel is required.

### INDUSTRIAL APPLICABILITY

According to the present disclosure, it is possible to achieve both good durable adhesive strength to enamel and color tone stability.

What is claimed is:

1. A dental adhesive kit comprising:
a dental adhesive composition (I) comprising
a polymerizable monomer (A1) having an acidic group which is a polymerizable monomer (A),
water (B),
a volatile organic solvent (C), and
a first photopolymerization initiator (D) comprising an aromatic tertiary amine compound (D1); and
a dental photocurable composition (II) comprising
the polymerizable monomer (A),
a second photopolymerization initiator (D′) comprising an α-diketone compound (D2), an iodonium salt compound (D3), and an aliphatic tertiary amine compound (D4) having an aromatic ring, and substantially free of the aromatic tertiary amine compound (D1), and
a filler (E).

2. The dental adhesive kit according to claim 1, wherein the dental adhesive composition (I) further comprises a compound (F) comprising a disulfide group.

3. The dental adhesive kit according to claim 2, wherein the compound (F) comprising a disulfide group is thioctic acid.

4. The dental adhesive kit according to claim 1, wherein the dental adhesive composition (I) is substantially free of an aliphatic tertiary amine compound.

5. The dental adhesive kit according to claim 3, wherein the dental adhesive composition (I) is substantially free of an aliphatic tertiary amine compound.

6. The dental adhesive kit according to claim 1, wherein the dental adhesive composition (I) comprises,
with respect to 100 parts by mass of the dental adhesive composition (I),
3 to 20 parts by mass of the polymerizable monomer (A1) having an acidic group,
10 to 50 parts by mass of the water (B),
10 to 60 parts by mass of the volatile organic solvent (C), and
0.05 to 2 parts by mass of the aromatic tertiary amine compound (D1), and
the dental photocurable composition (II) comprises,
with respect to 100 parts by mass of the polymerizable monomer (A) in the dental photocurable composition (II),
0.05 to 0.6 parts by mass of the α-diketone compound (D2),
0.3 to 5 parts by mass of the iodonium salt compound (D3),
0.5 to 5 parts by mass of the aliphatic tertiary amine compound (D4) having an aromatic ring, and
10 to 400 parts by mass of the filler (E).

7. The dental adhesive kit according to claim 3, wherein the dental adhesive composition (I) comprises,
with respect to 100 parts by mass of the dental adhesive composition (I),
3 to 20 parts by mass of the polymerizable monomer (A1) having an acidic group,
10 to 50 parts by mass of the water (B),
10 to 60 parts by mass of the volatile organic solvent (C), and
0.05 to 2 parts by mass of the aromatic tertiary amine compound (D1), and
the dental photocurable composition (II) comprises,
with respect to 100 parts by mass of the polymerizable monomer (A) in the dental photocurable composition (II),
0.05 to 0.6 parts by mass of the α-diketone compound (D2),
0.3 to 5 parts by mass of the iodonium salt compound (D3),
0.5 to 5 parts by mass of the aliphatic tertiary amine compound (D4) having an aromatic ring, and
10 to 400 parts by mass of the filler (E).

8. The dental adhesive kit according to claim 4, wherein the dental adhesive composition (I) comprises,
with respect to 100 parts by mass of the dental adhesive composition (I),
3 to 20 parts by mass of the polymerizable monomer (A1) having an acidic group,
10 to 50 parts by mass of the water (B),
10 to 60 parts by mass of the volatile organic solvent (C), and
0.05 to 2 parts by mass of the aromatic tertiary amine compound (D1), and
the dental photocurable composition (II) comprises,
with respect to 100 parts by mass of the polymerizable monomer (A) in the dental photocurable composition (II),
0.05 to 0.6 parts by mass of the α-diketone compound (D2),
0.3 to 5 parts by mass of the iodonium salt compound (D3),
0.5 to 5 parts by mass of the aliphatic tertiary amine compound (D4) having an aromatic ring, and
10 to 400 parts by mass of the filler (E).

9. The dental adhesive kit according to claim 5, wherein the dental adhesive composition (I) comprises,
with respect to 100 parts by mass of the dental adhesive composition (I),
3 to 20 parts by mass of the polymerizable monomer (A1) having an acidic group,
10 to 50 parts by mass of the water (B),
10 to 60 parts by mass of the volatile organic solvent (C), and
0.05 to 2 parts by mass of the aromatic tertiary amine compound (D1), and
the dental photocurable composition (II) comprises,
with respect to 100 parts by mass of the polymerizable monomer (A) in the dental photocurable composition (II),
0.05 to 0.6 parts by mass of the α-diketone compound (D2),
0.3 to 5 parts by mass of the iodonium salt compound (D3),
0.5 to 5 parts by mass of the aliphatic tertiary amine compound (D4) having an aromatic ring, and
10 to 400 parts by mass of the filler (E).

10. The dental adhesive kit according to claim 1, further comprising a dental etching agent.

11. A method for applying the dental adhesive kit according to claim 1, the method comprising
applying the dental adhesive kit while performing at least one of (i) splinting mobile teeth and (ii) orthodontic treatment for bonding to enamel.
